SEQUENCE LISTING (33 sequences)

SEQ ID NO: 1 - DNA, 1338 bp, L. acidophilus
atgaagaaaaatttaagaatcgttagcgctgctgctgctgctttacttgctgttgctccagttgctgcttctgctgtatctactgttagcgctgctactactattaacgcaagttcatcagcaatcaataccaacactaatgctaagtacgatgttgatgtaactcctagtgtttctgcagttgctgcaaatactgctaacaacactccagctattgccggtaaccttactggtactatttcagcaagttacaatggtaagacttatactgctaacttaaaggcagatactgaaaatgccactattactgctgctggtagcactactgccgttaaacctgctgaattagctgcaggtgtggcttacactgtaactgttaacgatgtttcatttaacttcggttcagaaaatgcaggtaagactgttacccttggttcagctaactcaaatgtaaaattcaccggtacaaacagtgataatcaaactgaaactaatgtttctactttgaaagttaagttagaccaaaacggtgttgcttcacttactaatgtttcaattgcaaacgtatacgcaattaacactactgataacagtaacgtaaacttctacgacgtaactagtggtgctactgtaactaacggtgccgtttcagttaatgctgataaccaaggtcaagttaatgttgcaaacgtagttgcagcaattaattcaaaatactttgcagcacaatacgcagataagaagttaaatactcgtactgctaatactgaagatgctattaaggcagccttaaaggaccaaaagattgatgtaaactcagtaggttacttcaaagcacctcatactttcactgttaacgttaaagcaacttcaaatactaatggtaagtcagctactttgccagtagttgttactgttcctaatgttgctgagccaactgtagccagcgtaagcaagagaattatgcacaacgcatactactacgacaaggacgctaagcgtgttggtactgacagcgttaagcgttacaactcagtaagcgtattgccaaacactactactatcaacggtaagacttactaccaagtagttgaaaacggtaaggctgttgacaagtacatcaacgctgcaaacatcgatggtactaagcgtactttgaagcacaacgcttacgtttacgcatcatcaaagaagcgtgctaacaaggttgtattgaagaagggtgaagttgtaactacttacggtgcttcatacacattcaagaacggccaaaagtactacaagatcggtgacaacactgacaagacttacgttaaggttgcaaactttagataataa

SEQ ID NO: 2 - protein, 413 aa, L. acidophilus
ATTINASSSAINTNTNAKYDVDVTPSVSAVAANTANNTPAIAGNLTGTISASYNGKTYTANLKADTENATITAAGSTTAVKPAELAAGVAYTVTVNDVSFNFGSENAGKTVTLGSANSNVKFTGTNSDNQTETNVSTLKVKLDQNGVASLTNVSIANVYAINTTDNSNVNFYDVTSGATVTNGAVSVNADNQGQVNVANVVAAINSKYFAAQYADKKLNTRTANTEDAIKAALKDQKIDVNSVGYFKAPHTFTVNVKATSNTNGKSATLPVVVTVPNVAEPTVASVSKRIMHNAYYYDKDAKRVGTDSVKRYNSVSVLPNTTTINGKTYYQVVENGKAVDKYINAANIDGTKRTLKHNAYVYASSKKRANKVVLKKGEVVTTYGASYTFKNGQKYYKIGDNTDKTYVKVANFR

SEQ ID NO: 3 - protein, 18 aa, Artificial Sequence
  insertion linker
RGSHHHHHHGSGTATTIN

SEQ ID NO: 4 - protein, 18 aa, Artificial Sequence
  insertion linker
AIAGNAMAAARGNLTGTI

SEQ ID NO: 5 - protein, 18 aa, Artificial Sequence
  insertion linker
VKFTGAMAAARAGTNSDN

SEQ ID NO: 6 - protein, 18 aa, Artificial Sequence
  insertion linker
TNVSITMAAARGIANVYA

SEQ ID NO: 7 - protein, 18 aa, Artificial Sequence
  insertion linker
YDVTSAMAAARASGATVT

SEQ ID NO: 8 - protein, 18 aa, Artificial Sequence
  insertion linker
NAANIVAMAAARGDGTKR

SEQ ID NO: 9 - protein, 18 aa, Artificial Sequence
  insertion linker
PSVSASMAAARGAVAANT

SEQ ID NO: 10 - protein, 17 aa, Artificial Sequence
  insertion linker
LKADTMAAARNTENATI

SEQ ID NO: 11 - protein, 18 aa, Artificial Sequence
  insertion linker
AELAASMAAARGAGVAYT

SEQ ID NO: 12 - protein, 18 aa, Artificial Sequence
  insertion linker
KTVTLAMAAARGLGSANS

SEQ ID NO: 13 - protein, 18 aa, Artificial Sequence
  insertion linker
NADNQAMAAARGQVNVAN

SEQ ID NO: 14 - protein, 23 aa, Artificial Sequence
  insertion linker
TTINAEQKLISEEDLNLESSSAI

SEQ ID NO: 15 - protein, 19 aa, Artificial Sequence
  insertion linker
AMVNEQKLISEEDLNARGN

SEQ ID NO: 16 - protein, 19 aa, Artificial Sequence
  insertion linker
AMVNEQKLISEEDLNARAG

SEQ ID NO: 17 - protein, 19 aa, Artificial Sequence
  insertion linker
TMVNEQKLISEEDLNARGI

SEQ ID NO: 18 - DNA, 39 bp, Artificial Sequence
  synthetic primer
gcgcgaattcagatctatcgtggtaagtaataggacgtg

SEQ ID NO: 19 - DNA, 72 bp, Artificial Sequence
  synthetic primer
cagcgaattcctcgaggtttaaatcttcttctgaaattaacttttgttctgcgttaatagtagtagcagcgc

SEQ ID NO: 20 - DNA, 38 bp, Artificial Sequence
  synthetic primer
ggggggatccggtaccgctactactattaacgcagaac

SEQ ID NO: 21 - DNA, 40 bp, Artificial Sequence
  synthetic primer
ccccggatccaagcttatcgaagtatcagaagatcctatt

SEQ ID NO: 22 - DNA, 39 bp, Artificial Sequence; identical to SEQ ID NO: 18
  synthetic primer

SEQ ID NO: 23 - DNA, 31 bp, Artificial Sequence
  synthetic primer
ggggaagcttcagtagtgctaccagcagcag

SEQ ID NO: 24 - DNA, 19 bp, Artificial Sequence
  synthetic primer
cttgctatttcttgaagag

SEQ ID NO: 25 - DNA, 21 bp, Artificial Sequence
  synthetic primer
gcgtttaaatcttcttctgaa

SEQ ID NO: 26 - DNA, 39 bp, Artificial Sequence
  synthetic primer
ggggggatccggtaccgctactactattaacgcaagttc

SEQ ID NO: 27 - DNA, 48 bp, Artificial Sequence
  synthetic primer
ccccgaattcaagcttttattagtatacgtttgcaattgaaacattag

SEQ ID NO: 28 - DNA, 37 bp, Artificial Sequence
  synthetic primer
ccccaagcttttattatgaagcaacaccgttttggtc

SEQ ID NO: 29 - DNA, 37 bp, Artificial Sequence
  synthetic primer
ccccaagcttttattaaccaagggtaacagtcttacc

SEQ ID NO: 30 - DNA, 41 bp, Artificial Sequence
  synthetic primer
ggggggatccggtaccaaagttaagttagaccaaaacggtg

SEQ ID NO: 31 - DNA, 41 bp, Artificial Sequence
  synthetic primer
ggggggatccggtacccttactaatgtttcaattgcaaacg

SEQ ID NO: 32 - DNA, 39 bp, Artificial Sequence
  synthetic primer
ggggggatccggtacctcagctaactcaaatgtaaaatt

SEQ ID NO: 33 - DNA, 46 bp, Artificial Sequence
  synthetic primer
ccccgaattcaagcttttattaaattctcttgcttagctgggctac

(19) United States
(12) Patent Application Publication
(10) Pub. No.: US 2010/0172938 A1
(43) Pub. Date: Jul. 8, 2010

(54) MODIFIED BACTERIAL SURFACE LAYER PROTEINS

(75) Inventors: Pieter Hendrik Pouwels, Rijswijk (NL); Egbert Smit, Sittard (NL); Frans Tielen, Prinsenbeek (NL)

Correspondence Address: NIXON & VANDERHYE, PC, 901 NORTH GLEBE ROAD, 11TH FLOOR, ARLINGTON, VA, 22203, US

(73) Assignee: NEDERLANDSE ORGANISATIE VOOR TOEGEPAST- NATUURWETENSCHAPPELIJK ONDERZOEK TNO, Delft (NL)

(21) Appl. No.: 12/648,271

(22) Filed: Dec. 28, 2009

Related U.S. Application Data
(63) Continuation of application No. 10/500,307, filed on Nov. 22, 2004, now abandoned.

(30) Foreign Application Priority Data
Dec. 28, 2001 (EP) 01310937.6

(51) Int. Cl. A61K 39/02; C07K 14/195; C07K 14/335; C07K 7/08; C07H 21/00; C12N 15/74; C12N 1/21; C40B 40/10; C12P 21/00; A61P 31/04; A61P 37/04

(52) U.S. Cl. 424/234.1; 530/350; 530/326; 530/325; 530/324; 536/23.7; 435/320.1; 435/252.3; 506/18; 435/69.1

(57) ABSTRACT

Modified bacterial surface layer (S-layer) proteins are disclosed where the modification is the insertion, at an internal location, of a heterologous polypeptide, or polypeptide of interest. The polypeptide is a binding or target protein, such as an antigen or antibody, or part thereof, in particular a bacterial antigen (e.g. from Clostridium tetani such as TTFC). The modified surface layer protein can then be expressed on the surface of the bacterial cell and used in a vaccine. Also disclosed are bacteria which have been modified to express a heterologous surface layer protein, but which do not as a wild-type possess an S-layer (such as L. casei), and modified bacteria which express only a modified surface layer protein (and not the wild-type S-layer protein). The wild type S-layer is completely replaced with the modified version where the polynucleotide encoding the modified version is integrated into the bacterial genome. The modified S-proteins can form crystalline arrays, sheets or layers that can be used to bind functional molecules (e.g. receptors) to solid surfaces (Au, silicon wafers) in biosensors.

### INTRODUCTION

The present invention relates to modified bacterial surface layer (S-proteins) that can be used in vaccines (when expressed on the surface of a cell), molecular sieves and sensors, and bacteria expressing such S-proteins. The S-layer proteins have inserted into them, at an internal position, one or more heterologous (or functional) polypeptides. Such a polypeptide may be an antigen or an antibody, or a portion thereof. Other modifications include the truncation of the carboxy (C) or amino (N) terminus to form fragments.

### BACKGROUND

Bacterial surface layer proteins are known to exist in many different types of bacteria, and often assemble into crystalline layers at the cell surface. However, the exact function of these proteins is not always known, although they are thought to play a role in bacterial adhesion to other cells, or to act as a scaffold for enzymes.

Lactobacillus is one of the genera belonging to the group of lactic acid bacteria (LAB) which are used in the manufacture of fermented foods and beverages, and are generally considered to be safe. Bacteria of the genus Lactobacillus can protect humans against infection, and this property (and their ability to colonise mucosal surfaces) has resulted in the use of Lactobacilli as vaccine delivery vehicles for oral immunisation. The adhesion to the mucosa is thought to be a pre-requisite for the survival and establishment in the gastrointestinal tract (GIT). Some surface located molecules have been identified as adhesions that specifically interact with receptor moieties in the internal tissue.

The surface layer of proteins self-assembles into multimeric units to form an array covering the entire cell as the outermost envelope. They are single protein molecules and form crystalline monolayers. However, the adhesive properties of S-layers, especially in probiotic Lactobacilli, remains poorly characterised. S-layers have been found in several Lactobacillus species, and although the function of these S-proteins is not known, they are thought to play a role in cell adhesion. S-layer proteins of Lactobacillus are amongst the smallest S-proteins known, and are different from most other bacterial S-proteins because they are highly basic (pI of at least 9) instead of acidic proteins.

Several S-layer proteins have been sequenced. These include the rsaA gene of C. crescentus (U.S. Pat. No. 5,500,353, Smit et al), and Lactobacillus acidophilus (Boot et al, 1993 and Smit et al, 2001). Although the sequence of the S-protein is given, the secondary and tertiary structure of the protein cannot be predicted simply from knowing the amino acid sequence. Indeed, from that document it is impossible to predict, for example, which amino acid sequence might be exposed to the surface, and which amino acids might be present in the S-layer.

In addition, the cbsA gene encoding the collagen binding S-layer protein of Lactobacillus crispatus has been expressed in L. casei. (Martinez et al, Journal of Bacteriology 182 (23): 6857-6861, December 2000).

As the structure and role of surface layer proteins is not well understood, there is therefore a need to determine a structure/function relationship, in order to allow the production of modified surface layer proteins which have improved properties over the wild-type. Surface layer proteins represent a large proportion of protein in the cell (as much as 10 to 12%) and may therefore be used to express large quantities of a heterologous protein. In addition there is a need for more effective (oral) vaccines, in particular those based on bacteria (or Lactobacillus) where a greater amount or higher density of antigen can be presented on the cell, or may be better presented at the cell surface.

### SUMMARY OF THE INVENTION

A first aspect of the present invention therefore relates to a (e.g. modified) bacterial surface layer (S-layer) protein, the modification comprising the insertion, internally, of a heterologous polypeptide. Such a polypeptide may be a functional one, and can be any polypeptide of interest.

The modification is the insertion of the polypeptide at an internal location. That is to say, the insertion is at an internal sequence, which is of course different from the addition (or fusion) of a polypeptide at either the carboxy (C) or amino (N) terminus. The insertion may take place at least 5, 10, 20, 30 or 40 amino acids from either end of the protein.

The polypeptide may be inserted (in the sense that it slots in between two adjacent amino acid residues) rather than replacing any residues (where residues are deleted or substituted).

### Location of Inserted Heterologous Polypeptide

The polypeptide may be inserted at a location whereby the polypeptide is exposed, or away from the cell surface when the surface layer protein is expressed (at the surface) of a bacterial cell. The heterologous polypeptide may therefore be exposed in the sense that it can be targeted or bound by a compound or other polypeptide (for example an antibody). This may be possible if the polypeptide is in a portion of the S-layer protein which is involved in or responsible for crystallisation and/or assembly, for example is part of the crystallisation or assembly domain.

However, the alternative is possible, where the heterologous polypeptide is not exposed, for example it is located (at least partly in) in the cell surface, or in the S-layer. This may be possible by inserting the heterologous peptide at a location at or near a cell wall anchor or is in an attachment or binding domain. This part of the molecule may be in the S-layer. In such case the heterologous peptide may be present or buried in the surface layer. It may thus be protected from the outside environment, for example shielded from proteolytic processing. It may not be bound or recognised by external antibodies or shielded from external degradation of proteolytic attack. This may be advantageous if the protein is to be located on a bacterial cell wall, and then administered to a human, as it may mitigate or avoid degradation, or proteolytic processing, by enzymes in the gastrointestinal tract (GIT).

For the S-layer protein of Lactobacillus acidophilus, two main domains have been identified. The first N-terminal region, which constitutes about two thirds of the molecule, is involved in crystallisation and assembly (and is called SAN). It constitutes amino acids 1 to 290 and effectively forms the S-layer above the cell wall. The second main domain, or last third of the molecule, amino acids 290 to 412, represents the cell wall anchor (and is called SAC). This portion of the molecule is buried in the S-layer.

The N-terminal assembly region can in itself be split into three main portions; 1 to 114 (involved in forming dimers and trimers); 150 to 290 (involved in forming dimers); these flank an intervening domain (about 115 or 120 to 150 or 160) that appears to be loop region (or a surface (immunodominant) loop) that is exposed and can be targeted by antibodies.

Therefore, if the heterologous polypeptide is to be exposed, then the insertion can be at a location of from any of amino acids 1 to 290, and preferably from 115 or 120 to 150 or 160 (as this appears to be a surface exposed loop region). The N-terminal crystallisation section or assembly region may be primarily hydrophobic, such as in L. acidophilus. This is unexpected, and counter-intuitive, given that this is a region that is (surface) exposed, and present in the environment outside the bacteria, which is necessary in order for the S-layer proteins to assemble and form a layer.

The cell wall anchor may be equally unusual, in that it is primarily hydrophilic, despite being present in the S-layer. (Cell wall binding regions are usually hydrophilic as they bind negatively charged groups in the cell wall). The cell wall anchor region can alternate between hydrophilic and hydrophobic regions in some S-proteins. The protein may thus be inserted in a hydrophobic region (e.g. for surface exposure) even though such a region would not usually be exposed on the surface.

The above two paragraphs relate mainly to Lactobacillus proteins, such as from L. acidophilus. For many other (S-layer) proteins the reverse is true, and in this case the N-terminal domain can be hydrophilic while the C-terminal may be hydrophobic.

If the heterologous polypeptide is to be exposed, then preferably it is located at a position from 100 to 150 or 160, such as from 110 to 140 or 150, preferably from 120 to 130 or 140, optimally at around position 125. Preferably the polypeptide is located in a region of the S-layer protein that is exposed or is in a surface (or immunodominant) loop.

Note that the numbering of residues in this specification is (unless the context requires otherwise) based on the numbering of the original, (mature) wild-type, protein. Another region for exposure of the heterologous protein is from 130 to 180, such as from 140 to 170, preferably from 150 to 160, optimally at around position 156. An additional location for exposing the polypeptide, although to a lesser degree, is at one of the positions from 20 to 80, such as from 30 to 70, preferably from 40 to 50, optimally at around position 45.

If the heterologous polypeptide is not to be exposed, or is to be at least partially present inside the S-layer, then the insertion can take place at a different location in the unmodified S-layer protein. This may be from position 1 to 30, such as from 3 to 20, preferably from 5 to 10, and optimally at about position 7.

Suitably the heterologous polypeptide is inserted into the protein, rather than replacing any part of the protein sequence. In that way, most of (eg. at least 70, 80, 85, 90 or even 95%) or the entire or full sequence of the (wild-type or original) protein can be retained. Hence, there may be no deletion or replacement of amino acid residues (or if there are, there are no more than 5, 10, 15 or 20 deletions or replacements). This may help the modified protein to retain as many of the properties or functions of the original molecule as possible. The modified protein may be at least as long, if not longer, than the unmodified (wild-type) protein.

### Modified Proteins

The following comments relate to both the modified (of the invention) and unmodified (wild type) proteins, unless the context requires otherwise. The protein preferably has a minimum size of 30, 40 or 50 kDa. It may have a maximum size of 70, 80, 100, 150 or even 200 or 300 kDa. Preferred proteins are from 40 to 50, 60, 70 or 80 kDa in size. The protein may be able to crystallise, or with other proteins form a sheet or crystalline monolayer (e.g. in vitro). They may be able to self-assemble, for example into multimeric units. If possible, the protein may be able to form (with other proteins) an oblique lattice (for example of p1 or p2 symmetry).

The protein may have at least two domains or functional regions. One may be involved in or responsible for crystallisation or assembly. This region may be exposed, and away from the cell membrane. The protein may therefore have a (e.g. surface exposed) loop region. The protein may have a cell wall anchor or cell wall binding region. This may be wholly or partly present in the S-layer.

Preferably the modified protein will not comprise (or retain) a (eg. S-layer) secretion signal (this may have been cleaved during processing or expression). It may have a (C-terminal or cell wall) anchor region, which may be the original region from the S-layer protein or it may be (in addition) such a region from another S-layer protein, such as of a different species (preferably from the species in which the modified protein is to be expressed).

The (unmodified or wild-type) protein may be from a lactic acid bacteria, such as from Lactobacillus. Preferably, the protein is from L. acidophilus, L. crispatus, L. helveticus, L. amylovorus, L. gallinarum and/or L. brevis. Preferably the protein is predominantly basic. Suitably, the protein is not from a Gram negative or aquatic bacteria, for example of the group Caulobacter, such as from C. crescentus, or from the family Campylobacteraceae (eg. those belonging to Campylobacter).

The protein may be expressed in a bacterial host. If it is expressed on the surface, such as in the S-layer, then bacterial host cell may be used in a vaccine. The heterologous polypeptide may be exposed, e.g. to the environment. If however the polypeptide is to survive inside the body (or be shielded from the environment or from degradation or proteolytic attack), for example in part of the GIT, then the heterologous peptide may be partially or wholly present or buried in the S-layer.

Preferably the protein has a pI of at least 6, 7 or 8. Optimally however it has a pI of at least 9, such as at least 9.4.

### Insertion Sites

The location of the inserted polypeptide may effect crystallisation. Modified peptides which crystallise, or do not crystallise, can both be prepared using the invention. The position of insertion of the polypeptide may be at from 1 to 290, such as at any of the following locations (if for example the protein is desired to be able to crystallise):
(1) at from 1 to 20, such as 3 to 15, preferably from 5 to 10, optimally at about position 7;
(2) at from 35 to 55, such as 40 to 50, optimally at about position 45;
(3) at from 100 to 130, such as from 110 to 120, optimally at about position 114; and/or
(4) at from 110 to 140, such as from 120 to 130, optimally at about position 125.

The heterologous polypeptide can be inserted at position above residue 290, such as from 291 to 400, 425 or 450 (eg. about 412). If the protein is not to be capable of crystallising then the insertion may be:
(1) at from 20 to 40, such as 25 to 35, optimally at about position 30;
(2) at from 50 to 80, such as at 60 to 70, optimally at about position 66;
(3) at from 70 to 100, such as from 80 to 94, preferably from 85 to 90, optimally at about position 88; and/or
(4) at from 115 to 150 or 140 to 180, such as from 150 to 170 or 160 to 180, preferably from 150 to 160 or 170 to 180, optimally at about position 156 or 177.

An insertion at above position 290 may not affect crystallisation as this may not be in a surface or exposed position (for example it may be in a cell wall anchor region). The insertion may thus be:
(5) at from 320 to 410, such as from 330 to 370, preferably from 340 to 360, optimally at about position 349.

There may be one or more heterologous polypeptides, inserted at one or more different sites.

### Sensors and Molecular Sieves

The protein or bacterial host cell can be employed in sensors, for example included in a coating. They may also find use in molecular sieves. For example, a plurality of bacteria, expressing a modified protein, can be arranged to form a hole or to define a pore. By changing the location and nature of the heterologous polypeptide, one can change not only the protein size and characteristics but also the confirmation of the protein. This is turn may change the pore or hole characteristics, for example in certain parameters such as affinity or charge.

A multiplicity of proteins may be able to crystallise or form 2-dimensional arrays on a (e.g. solid) surface (e.g. comprising silicon, polymer(s) or metal(s)) or an interface (e.g. a liposome or planar lipid film). An S-layer may be used as a template in the formation of an inorganic (e.g. nanocrystal) lattice (e.g. comprising CdS, Au, Ni, Pt or Pd), such as in electronics or non-linear optics.

The functional domains on each S-protein may be repeated with the periodicity of the S-layer lattice. This may allow the formation of regular arrays of bound molecules or particles.

The (modified) S-proteins of the invention may interact with each other, and if appropriate, with a supporting cell envelope layer (such as plasma membrane, outer membrane or peptidoglycan). The protein may have a high proportion of non-polar amino acids, so that hydrophobic interactions play a role in assembly. Preferably, a multiplicity of S-proteins have the capability to reassemble, for example in suspension, at a liquid air interface, at a solid surface, at a floating lipid monolayer or on (the outside of) a liposome. The S-proteins may therefore form a monolayer, at a surface or an interface, and this allows them to be used in nanobiotechnological applications.

A further aspect of the invention therefore relates to a sheet or (optionally crystalline) monolayer or two dimensional array comprising a polarity of bacterial surface layer proteins. At least one of such proteins is preferably a modified protein according to the first aspect.

The invention therefore stems, in a further aspect, to a solid surface, liquid-air interface, lipofilm, liposome or solution comprising a sheet, monolayer or array of the previous aspect.

Preferably, a multiplicity of the S-proteins may be disintegrated by a chaotropic agent, by lowering or raising the pH value, or by addition of a chelating agent or cation substitution. Preferably, after removal of the disrupting agent, the proteins will reassemble into a fiat sheet, an open-ended cylinder or a closed vesicle. Any of these arrays may be a double layer, or bi-layer, for example with two constituent monolayers facing each other (either with their inner or outer side). Bi-layers may, if necessary, contain divalent cations interacting with polar groups.

The S-proteins may be able to reassemble at an air-water interface, on a solid support, or in a lipid film. In this way, a (randomly aligned) monocrystalline S-layer may be formed. If formation takes place at a liquid-solid interface, then the solid may comprise silicon, such as a silicon wafer. The silicon wafer may have a native oxide layer, it may be silanised, or it may be coated with a photoresist and/or carbon film. Other solids can be employed, such as silicone nitride, galium arsenide, noble metals (gold or platinum), glass, cellulose, graphite or mica.

Preferably, the S-layer can be patterned using deep ultra-violet (DUV) radiation. The S-layer, such as on a silicon wafer, may be brought into (direct) contact with a photomask, and exposed to DUV radiation. The S-layer may be removed from the silicon wafer or substrate at the exposed areas. Thus, a pattern on the photomask may be transferred into the S-layer.

The S-layer protein (or sheet or array) may be used to immobilise a biologically active (macro) molecule. Molecules such as enzymes, antibodies, antigens and/or ligands may be covalently immobilised to an S-protein (mono) layer.

A layer of S-proteins may therefore act as a surface linking layer. The S-protein layer may be inbetween, or sandwiched between functional molecules and a solid surface or support. The functional molecule may be an enzyme, antibody (or fragment thereof), or other binding molecule, a receptor, antigen or ligand, any of which may act as a bioreceptor (such as for a target, in a sample). The S-layer proteins may be self-assembling, and they may be capable of recrystallising at the surface. They may thus provide a linking layer between the solid surface and the functional molecules. This type of arrangement may be used in a (bio) sensor, and the invention therefore relates to sensors, molecular sieves and ion traps comprising an S-protein layer of the invention.

### Fragments of Surface Layer Proteins

The invention additionally relates to modified proteins that are fragments of bacterial S-layer proteins. The fragments may be N-terminal fragments. Alternatively or in addition, the fragments may be capable of forming trimers, for example with two other such fragments. The fragment may be able to form multimers (e.g. timers) with different fragments, or indeed the wild type molecule as well. These fragments can be thought of as C-terminal deletions because, as they retain a portion of the N-terminus, they can be prepared by, in effect, deleting or removing all or a portion of the C-terminus.

The fragments may be capable of forming dimers, preferably with another such fragment (although the formation of dimers with other fragments or the full size wild-type molecule may also be possible). Such fragments may include the (immunodominant or exposed) loop region. The fragment may then be from 20 to 200 amino acids long. Another type of fragment may be one that excludes the entire (immunodominant or exposed) loop region. This may allow for at least part of the loop region to be present. Such fragments may be from 20, 30 or 40 to 100, 130 or 155 amino acids long.

The loop region may be a region that, in the full length of wild-type molecule, is exposed to the environment when expressed on the surface of a bacterium. It may be exposed in the sense that it may be bound or targeted by an (external) antibody specific for that region. The N-terminal fragments or those that are capable of trimerisation may include part or all of the loop region. The fragments may thus be from 10 to 30, 40 or 50 amino acids long. For certain bacteria, such as L. acidophilus, this region may be from 20 to 40 amino acids in length, such as from 32 to 38 amino acids.

Preferably the fragment is at least 40, 60, 80, 120, 130 or at least 140 amino acids in length. The fragments may be up to 170, 150, or 120 amino acids in length.

If the fragments are fragments of an S-layer protein from Lactobacillus, such as L. acidophilus, the fragments are suitably fragments of the assembly or crystallisation region, known as SAN in the art, namely residues 1 to 290. Particularly preferred fragments (using the numbering of the residues of L. acidophilus S-layer protein) are 1-159, 1-149, 1-114, 115-290, 139-290 and 150-290. Preferably the fragments will not contain any part of SAC or the cell wall anchor region.

### Novel (Bacterial) Cells

A second aspect of the invention relates to a (e.g. modified) bacteria, or bacterial cell, which has been modified to express a heterologous surface layer (S-layer) protein. Preferably, the bacterium is other than an L. casei cell or a Bacillus (e.g. B. sphaericus or B. brevis) cell. Preferably the bacterial cell is however Lactobacillus cell or from the family Lactobacillaceae. The S-layer protein may have its own (or original) cell wall anchor. In other words, the cell wall anchor is homogenous to the S-layer protein, or the protein retains its natural cell wall anchor domain, or region. Preferably, the S-layer protein has a cell wall anchor which is from the same bacterial species as the S-layer protein. Such a bacteria, as a wild-type, or in unmodified form, may not possess such a surface layer.

Preferably the (host) cell expresses, or contains a gene encoding, from 1 to 6 different surface layer proteins (either naturally, as a wild type, or in a modified form).

The S-layer protein is preferably one of the modified bacterial surface layer proteins of the first aspect, although it need not be. For example, the bacterial cell may be one which does not (normally) have an S-layer, (for example, Lactobacillus reuteri, Lactobacillus fermentum, Lactobacillus salivarius, Lactobacillus animalis, Lactobacillus gasseri, Lactobacillus johnsonii, Lactobacillus murinus, Lactobacillus sakei, Lactobacillus curvatus, Lactobacillus delbrueckii subsp bulgaricus, Lactobacillus delbrueckii subsp lactis, Lactobacillus plantarum, Lactobacillus pentosus, Lactobacillus casei, Lactobacillus zeae, Lactobacillus paracasei, Lactobacillus rhamnosus or a bacteria from Lactococcus) but in modified form does have an S-layer (or at least carries an (e.g. modified) S-protein at its surface). The S-layer may thus comprise a naturally occurring surface layer protein, albeit one that is heterologous to the bacteria. For example, the bacteria may be L. casei, which does not possess an S-layer, but which has been modified to express surface layer proteins from another bacteria, such as from a lactic acid bacteria or Lactobacillus, for example L. acidophilus.

A third aspect relates to an L. acidophilus cell which expresses (e.g. on the surface of the cell) a heterologous surface layer protein. In this case, L. acidophilus does naturally possess an S-layer, and so the S-protein may be a modified one, or one from a (bacterial) species other than L. acidophilus, or both.

The surface layer protein may thus be a naturally occurring or wild-type protein, or it may be a modified surface layer protein for example one of the present invention, as described in the first aspect, or a mixture of both. Preferably the S-layer protein is antigenic and/or induces an immune response. The protein is suitably not a part of a (e.g. collagen) binding surface layer protein, for example of L. crispatus.

Preferably the number of S-layer proteins is sufficient to form an S-layer, and preferably an S-layer that covers the entire cell. Hence the number of S-proteins at the cell surface may be at least 1, 2 or 5×10⁵ S-layer proteins (or monomers), preferably at least 10⁶, optimally at least 5×10⁶ monomers.

Preferably the S-layer protein, even if modified, retains the cell wall anchor of the unmodified or wild-type protein. However, it may have a heterologous anchor or binding region or domain, and so may be a fusion protein. This may attach the protein to the S-layer, cell wall, lipid bilayer, cytoplasmic membrane, peptidoglycan or to a cell wall component. Whatever form of linkage, the protein is preferably located at or in the surface of the host.

Four different types of cell wall binding domains are envisaged. The first may bind (e.g. covalently) to the peptidoglycan part of the cell wall. Suitable anchor may be from a proteinase, such as PrtP (Novarre, 1994). Secondly, the binding may be (e.g. covalently) to the lipid bilayer, for example through N-acyl diglyceride modification of a cysteine residue (for example located immediately C-terminal to the signal sequence cleavage site (Pugsley, 1993). Thirdly, there may be a non-covalent linkage to the cytoplasmic membrane (for example, Lysteria monocytogenes actA, Cocks et al., 1992). A fourth possibility is an anionic interaction, such as non-covalent one, which may be to a cell wall component. This may thus be a different method from linkage to a peptidoglycan or membrane.

For the first type of linkage, the fusion protein may comprise a sorting signal (such as from a proteinase (PrtP) or L. casei). Preferably the bacteria heterologously expresses an S-layer protein encoding gene to produce the expressed protein.

A fourth aspect of the invention relates to an L. casei bacterial cell expressing a (bacterial) surface layer protein that is not from L. crispatus and/or is not a collagen binding protein. The S-layer protein may be one that does not bind collagen, for example it is not a collagen binding S-layer protein. Preferably the S-layer protein is antigenic, or capable of inducing an immune response. Preferably, the L. casei cell expresses an S-layer protein that is naturally from, or originates from, L. acidophilus. However, the S-layer protein may be derived from a wild-type or naturally occurring L. acidophilus protein, and for example it may be an S-layer protein modified in accordance with the first aspect. The S-protein may be expressed on the surface of the cell. This may be achieved by providing the S-protein with a cell wall anchor that allows it to bind to the cell-surface of L. casei, for example an anchor region from an L casei surface layer protein. This may be from a proteinase (such as PrtP).

A fifth aspect of the invention relates to a (modified) bacteria expressing only, or homogenously, a heterologous or modified bacterial surface layer (S-layer) protein. The protein may be heterologous in the sense that the bacteria does not normally or in unmodified form does not express that protein. The heterologous or modified protein may therefore replace the natural or wild-type protein, preferably completely, or in its entirety. Thus, the bacteria may express only the heterologous or modified S-layer protein, but not any of the wild-type S-layer protein, at least in the S-layer. (Previous aspects included, where appropriate, cells that expressed both the wild-type S-protein as well as a modified S-protein). The bacteria may be another than from Caulobacter.

The fifth aspect therefore includes bacteria having a genome which contains or incorporates a polynueleotide encoding a heterologous (to the bacteria) or non-wild-type or modified S-layer protein. That is to say, such a polynucleotide may be integrated into the genome. Preferably, the gene or polynucleotide encoding the wild-type or natural S-layer protein has been silenced, switched off, replaced or otherwise caused to be non-expressed. The wild-type may thus be exchanged for a mutant (allele), in effect in vivo.

Preferably the surface layer forms an array, and may cover the entire cell. It may thus form an outermost envelope. The surface layer may be a crystalline monolayer, or comprise (e.g. self) assembled S-layer proteins. These may be in the form of monomeric units. The S-layer may therefore comprise an array of single protein monomers (the S-proteins).

In all aspects, suitably the S-layer protein, or bacterial cell, is from a lactic acid bacteria, or from the Bacillus/Clostridium group of bacterial (e.g. low GC, Gram positive bacteria). Suitably the protein or cell will be from the family Lactobacillaceae, such as genus Lactobacillus.

### Bacterial Cells of the Invention

The cell may be a Gram positive bacteria, for example from the AT rich (rather than GC rich) group (also called the low GC class). Suitably the bacteria is from the Actinomycetes class (which includes Carnobacterium, Lactobacillus, Lactococcus, Leuconstoc, Pediococcus and Streptococcus. Preferably it is non-aquatic. It may be a Lactobacillus cell, or a lactic acid bacteria (LAB). It is preferably L. plantarum (such as L. plantarum 80 or 256), L. casei, L. brevis, L. acidophilus, L. crispatus, L. helveticus, L. amylovorus, L. gallinarum or L. lactis. Preferably the host cell is one that can express the desired modified protein, which may be antigenic, for example using recombinant techniques. The host cell may express only the modified S-layer protein (and no wild-type S-layer protein, as in the third aspect above) or it may express both (the modified and unmodified) S-layer proteins.

A Lactobacillus plantarum strain can readily be determined using known parameters (e.g. Bergeys Manual of Determinative Bacteriology and Vescovo et al, Ann. Microbiol. Enzymol. 43 261-284 (1993)). The skilled person can therefore readily determine whether a lactic acid bacterium is a Lactobacillus plantarum. Numerous Lactobacillus plantarum strains have been deposited at various institutes and are readily available.

The bacterial (host) cell should preferably have GRAS (generally regarded as safe) status, and more preferably be food grade. This may thus exclude soil bacteria, or Bacillus (e.g. food spoilage organisms, such as Bacillus terreus or sphaericus). Such organisms are not suitable for administration to humans (e.g. as a vaccine).

Also, the bacterial host used should allow—upon transformation with an appropriate construct encoding an antigen—expression of the modified protein on the surface.

Preferably the level of expression of the protein, e.g. intracellularly, secreted and/or exposed on the surface (such as determined by SDS-polyacrylamide gel electrophoresis or FACS), should be at least 0.1, 0.5, 1, 3 or 5, 7, 10 or 15% (e.g. 8, 9 or 10 to 12, 13 or 14%) of the total cell protein.

Also, the bacterial host is preferably capable of settling or persisting in and/or colonizing at least part of the gastrointestinal tract, such as the mouth, the throat, the larynx, the gut, the small intestine, the large intestine, the ileum and/or the colon, vagina, or a combination thereof. Preferably the bacterial host is such that it mainly settles in the intestine, more preferably in the small intestine or caecum.

The bacteria preferably exhibits a persistence in the individual to be immunized (upon oral administration and as determined by the presence of the strain in the faeces) of at least 5 days, preferably at least 9 days, and suitably more than 15 or even 20 days. Longer persistence may not be required if an administration regimen comprising the use of one or more booster immunisations as described below.

The L. plantarum strain may be LMG 9211 (NCIMB 8826 as described by Mercenier above), DSM 4229 L. casei 393, and/or L. plantarum 80. Preferably however the bacterial host is one of the following L. plantarum strains: 256, LMG 1284, LMG 6907, LMG 8155, LMG 9205, LMG 9206, LMG 9208, LMG 9209, LMG 9210, LMG 9212, LMG 11405, LMG 11460, LMG 8095, LMG 8027, LMG 12167, LMG 13556, LMG 17552, LMG 18021, LMG 18023, LMG 18024, LMG 18027, LMG 18095; 386, 299, 105 or 275 (see Molin et al., 1993. J. Appl. Bacteriol. 74:314), 299v (see WO 96/29083); So5,36^{E}, 95, 120 or 44 (see Johannson et al 1995, Int. J. Syst. Bacteriol, Vol 45(4):670-675), 79, 107, 98, 53, 97, 101 or 125 (see Johansson et al 1995 Int. J. Food. Micro. 25:159), CH, ATCC 8041, ATCC 10012, ATCC 10776, WCFS, DF66 IIIa, DF66spez.-IVa, and/or the L. plantarum strains available from the Japanese Collection of Micro-organisms under the accession numbers: 8341, 8342, 8343, 8344, 8345, 8346, 8347 and/or 8348.

Preferably the (e.g. Lactobacillus) bacterium employed is foreign to the individual (human or animal) to which it is to be administered, for example vaccinated. The bacteria of the invention are modified or recombinant, and it is so the wild-type or naturally occurring bacteria, that is to say the non-recombinant or unmodified bacteria, that is suitably foreign. Thus by foreign it is intended to refer to a (e.g. Lactobacillus) strain not normally present in the individual. In other words, it is preferred to use an (e.g. Lactobacillus) strain that is non-human, for example it is not found or present in humans. Preferably the strain will be one not found in humans or animals, for example a strain that does not exist in the gut (G.I. tract) and/or vagina. Such strains will provoke a greater or larger immune response. Although some prior workers have used human-derived L. plantarum strains (such as NCIMB 8826, a human saliva isolate) it is preferred to use a strain not present in or originating from humans.

The present invention therefore preferably uses a bacterial strain that is not found in the mucosa (non-mucosal) or G.I. tract of individual, for example it is not endogenous (to humans or a species of animal to be vaccinated). Indeed, the most preferred strains (e.g. L. plantarum 256) are found in silage. Such strains are clearly foreign to humans, and so provide an increased immune response. Nevertheless, preferably the strain may be capable of adhering to the intestinal mucosa.

The strain may also be of non-foodstuff origin, for example not found in (human or animal) foods. This may thus exclude some L. casei strains, e.g. 393. Strain 393 is found in cheese, and so preferably strains found in dairy or fermentation products are excluded. This means that the individual has not (or may not) have had contact (e.g. ingested) the strain, and so using strains not found in foods is more likely to provoke an immune response. This may explain the long persistence times found with certain Lactobacilli. It may be that those bacteria are cleared more slowly as they are not recognised by the immune system. Suitably the strain is thus of animal origin, for example from an animal feedstuff (e.g, silage). Strains that are generally more suitable are commensal (in the gut), rather than dietary (e.g. dairy origin).

Preferably the bacterial (e.g. Lactobacillus) strain is viable (or alive) and intact. Suitably it will be able to persist (in the mucosa) of the individual for at least 7 days. This can easily be tested using procedures known in the art (for example, testing for the existence of the organism in faeces).

Thus the cell may be a non-human and/or non-human food Lactobacillus bacterium, such as L. plantarum, which has been modified to express the modified protein intracellularly or on the cell surface. This bacterium is preferably able to elicit an immune response in an individual, to whom the bacterium is administered. Thus the naturally occurring or unmodified L. plantarum is preferably foreign to that individual, for example it is not endogenous to humans or the animal to which it is to be administered. Preferably the chosen L. plantarum strain will not be present in the G.I. tract or mucosa of humans or that species of animal.

Other suitable strains may be selected by the skilled person on the basis of one or more of the following properties or factors:
stability of the construct encoding the antigen in the bacterial host selected; level of expression of antigen in the bacterial host selected; regulation of expression of antigen in the bacterial host selected; site of expression of antigen in the bacterial host selected; and/or stability of antigen produced;
the biochemical properties of the strain used, for example its sugar fermentation profile (API), cell wall composition, structure of LTA, structure of peptidoglycan, 16S RNA sequence, acid resistance, bile acid resistance, agglutination properties, adjuvanticity, immune modulating properties, in vitro adherence properties, mannose-specific adherence, presence of proteinaceous adherence factors, presence of mapA-like adherence factors and/or presence of large proteinaceous adherence factors with repeated amino acid sequences; and/or
the interaction of the bacterial host with cells of the individual to which the host to be administered (i.e. as part of a vaccine according to the invention) including but not limited to its persistence, viability, in vivo expression of antigen and/or tissue-specific persistence.

In at least some, preferably most, and more preferably essentially all of these properties, the strain used should be essentially (at least) equivalent to the strains mentioned above, and more preferably equivalent to L. plantarum 256, e.g. as determined on the basis of tests/assays for these properties known per se in the art. After a suitable host has been selected, it may be transformed with a genetic construct as described herein, after which its suitability as an oral vaccine may be tested. It is envisaged that on the basis of the description herein, and optionally after carrying out—for the purposes of confirmation—the tests described herein, the skilled person will be able to identify other L. plantarum strains suitable for use as or in vaccines of the invention.

Preferably the L. plantarum is in the group (or cluster) 1, (which includes strains 101, 97, 53, 256, ATCC 14917, 36^{E}, 95, 98, 299, 299v, 107, 105, 79, 275, 386, So5 and ATCC 8014), suitably subgroup (or subcluster) 1b (which includes 256, ATCC 14917, 36^{E}, 95 and 98), rather than subgroup (or subcluster) 1c (which covers human originating strains). Particularly preferred is the L. plantarum strain 256.

To classify L. plantarum strains a method can be used in which the genomes of the Lactobacillus strains are characterized by performing a restriction endonuclease analysis (REA) of chromosomal DNA followed by agarose gel electrophoretic separation of the fragments (Stahl et al, Int. J. Syst. Bacteriol. 40: 189-93 1990; Johansson et al, Int. J. Syst. Bacteriol. 45, 670-675 1995). The power of the REA technique is that it provides a unique fingerprint of the chromosomal DNA of each strain. When REA was applied to classify L. plantarum strains and the results were analysed by cluster analysis it was shown that this species is comprised of two clusters. Cluster 1 comprises strains originating from both human intestine and fermented food and feed, whereas cluster 2 comprises only two strains from Nigerian ogi. Cluster 1 contains 3 subgroups, 1a, 1b and 1c. All human L. plantarum strains were found in group 1b. L. plantarum strains of non-human origin were found in all 3 sub-groups.

A further distinction between L. plantarum strains from the three subgroups of cluster I was described by Adlerberth et al (Appl. Enviromn Microbiol. 62, 2244-2251 1996) who observed that strains from subgroup 1c adhered considerably stronger to human adenocarcinoma cells in vitro compared to strains from subgroups 1a and 1b. Strains from subgroups 1a, 1b and 1c can be further discriminated on the basis of their hemagglutination patterns. Strains from subgroup 1c strongly agglutinated human, guinea pig, chicken, cat dog, mouse, rabbit, rat, horse and pig erythrocyte, while those from 1a and 1b display strongly reduced or no agglutination characteristics.

A combination of one or more of the strains mentioned above may also be used.

Preferably the host cell has only a limited persistence in the gastrointestinal tract, such as from 3 to 10 days, preferably from 4 to 8 days, similarly from 5 to 7 days.

### BRIEF DESCRIPTION OF THE FIGURES

FIG. 1. (A) Linker insertion mutagenesis and E. coli expression vector constructions. Sites chosen for linker insertion are indicated by solid vertical arrows. For details on linker insertion positions the reader is referred to Table 1. (B) Lactobacillus vector constructions. Promoter sequences are indicated by an arrow pointing to the right and a stemloop indicates the presence of a transcription terminator sequence. Black barr indicates the slpA sequence encoding mature S_{A}-protein and grey bars the signal sequence. Restriction sites are abbreviated as follows: B, BamHI; K, KpnI; P, PstI; Bs, BstEII; H, HindIII, Nc, NcoI; N, NotI; X, XhoI, S, SalI; Bg, BglII; E, EcoRI.

FIG. 2. SDS-PAGE analysis of S_{A}-protein mutants purified from E. coli. Total (T), pellet (P) and supernatant (S) fractions obtained by dialysis and centrifugation. The numbers above each panel represent mutant numbers.

FIG. 3. SDS-PAGE and western analysis of surface protein extracts obtained from wild type L. acidophilus (lane 1) and transformants producing mutant S_{A}-proteins S_{A9c}, S_{A11c}, S_{A12c} and S_{A13c} (lanes 2 to 5). The top panel shows a coomassie stained SDS-PAA gel, the middle panel a western analysis with anti-S_{A}-protein and the lower panel a western analysis with anti-c-myc. The arrow indicates S_{A}-protein (43 kDa) and the Mr's of the reference proteins are given in the left margin.

FIG. 4. Detection of c-myc at the cell surface of L. acidophilus transformants using flow cytometry. Cells were coated with mouse monoclonal anti-c-myc antibodies followed by fluorescent labelling with goat-anti-mouse FITC-conjugated antibodies. Fluorograms of L. acidophilus producing S_{A9c}, S_{A11c}, S_{A12e} and S_{A13c} are indicated with 9c, 11c 12c and 13c, respectively. The inset shows the negative control (wild type L. acidophilus).

FIG. 5. Western analysis of SAN and four N- or C-terminally truncated SAN peptides. Peptides were separated by SDS-PAGE and detected with anti-his antibody. Oligomers of SAN ( [symbol] ) and dimers of SAN2, SAN4, SAN6 and SAN7 ( [symbol] ) peptides are indicated with arrows (lane 1 to 5, respectively). Mr's or reference proteins are indicated in the right margin.

FIG. 6. Hypothetical structural organisation of S_{A}-protein monomer and the L. acidophilus S-layer. (A) Contact points of one repeating unit with surrounding units are indicated with arrows and the putative boundary between two morphological subdomains (black and black shading, respectively) with a dashed line. (B) Overview of repeating units forming the S-layer structure. Repeating units as well as subdomains are indicated as in (A). (C) Schematic representation of one S_{A}-protein monomer showing the SAN and SAC domains and the two putative SAN subdomains. (D) Schematic representation of a cross-section of the L. acidophilus cell wall and S-layer.

### BRIEF DESCRIPTION OF THE SEQUENCES

SEQ ID NO. 1 is a DNA sequence encoding the (prior art) L. acidophilus (mature) S-layer protein (slpA gene) which was modified to create the surface layer proteins of the invention;

SEQ ID NO. 2 is the amino acid sequence of SEQ ID NO. 1;

SEQ ID NOS. 3 to 17 are amino acid sequences showing the position of (and including) the heterologous polypeptide (sequence) used to create the modified proteins of the invention; and

SEQ ID NOS. 18 to 33 are primers used to construct the proteins of the invention.

### Mode of Vaccine Delivery

Preferably this is a mucosal or oral or nasal vaccine. This may contain one or more of the bacterial cells of any of the aspects of the invention previously described. The vaccine may comprise lactose, such as to assist in the expression of the modified protein. This can mean any vaccine suited, adapted, intended and/or formulated for mucosal delivery.

Mucosal delivery means any route of administration to the body of a human or animal for which it is not required to penetrate or puncture the skin (e.g. as with intravenous, intramuscular, subcutaneous or intraperitoneal administration). Usually, this means that the vaccine is administered to the body via one of the body cavities, such that it comes into contact with the mucosa. Hence mucosal administration in particular refers to nasal, oral and/or vaginal administration.

Oral delivery (of a vaccine) means any route of delivery to the body of a human or animal into, by which the vaccine can be presented to, the gastrointestinal (G.I.) tract or any part thereof. Usually, this will involve administration into or via the mouth into the G.I. tract. This also includes administration directly into the G.I. tract or into any part thereof, and including into the stomach, for instance using a tube or catheter.

An oral vaccine is one that is suited, adapted, intended and/or formulated for oral delivery as defined above.

### Immune Response

Suitably the vaccine will elicit an (immune) response. A response (e.g. an antibody response or immune response) is deemed significant if it leads to a detectable change or response in a human or animal, and in particular to a detectable immunological change or response, such as the production of antibodies, cytokines, lymphokines, etc. Tests for determining whether a response is significant are known in the art and include, but are not limited to, titration of antibody levels in biological samples using ELISA techniques, ELISPOT techniques and in vitro lymphocyte stimulation assays. Such techniques are usually carried out on a biological sample, such as a biological fluid or cell sample, obtained from the human or animal.

A significant response may be, but is not necessarily, also a protective response as defined below. A response (e.g. an immunological response against a pathogen or an antigen) is deemed significant when it is capable of protecting the human or animal having the response against the pathogen and/or against a pathogen associated with the antigen.

An antigen is deemed exposed on a bacterial host (also referred to as (surface) exposition of the antigen) when it is present, forms part of, is attached to, and/or is otherwise associated with or detectable on (e.g. using a suitable immunological detection technique such as FACS or immunofluorescent microscopy) the surface of the bacterial host (e.g. the bacterial cell wall or envelope)

Preferably, exposed means that the bacterium, when presented to a cell of a human or animal that is capable of mediating an immune response (such as the cells of the G.I. tract mentioned below) for a sufficient time and in a sufficient amount, is capable of eliciting a sufficient immune response against the antigen.

### Vaccines

A preferred embodiment of the invention is a vaccine wherein the bacteria comprises an expression vector, for example capable of expressing the modified protein (e.g. an antigen), suitably on the cell surface (or intracellularly). The modified protein may thus be expressed internally or externally. Preferably the protein is exposed on the cell surface (e.g. under conditions present in the gastrointestinal tract).

The bacteria may express a modified protein (e.g. the heterologous polypeptide) specific for mucosa colonising pathogens or pathogens entering the body via the mucosa, specifically via the oral route. The heterologous polypeptide (e.g. antigen) may thus be specific for a gastrointestinal tract colonising pathogen. A heterologous polypeptide specific for tetanus (Clostridium tetanus), such as TTFC, is a particularly suitable candidate.

Preferably the vaccines are formulated such that a single dose is sufficient. However embodiments where multiple applications over a period of time, e.g. with a view to the persistence of the bacterial host in the G.I. tract, are also envisaged. The provision of booster vaccinations is also envisaged with the vaccine formulations according to the invention.

A preferred administration regimen comprises one or more “initial” doses or administrations on any of days 1 to 4, followed by one or more booster administrations on any of days 14 to 21, and optionally one or more further booster administrations on any of days 28 to 25. A single initial administration, followed by a single booster administration, within this time period, will generally be sufficient.

As high a degree of expression as possible without damaging the viability of the cell or the host to be vaccinated is envisaged. With higher expression, less frequent and lower doses may be required for immunisation purposes. Naturally the dosage regime will not only depend on amount of antigen but also on antigen type and the presence or absence of other immunogenicity stimulating factors in the vaccine.

One can use homologous expression and/or secretion signals in the expression vector(s) present in the recombinant bacteria in the vaccine: this may give a high degree of expression. The expression regulating signals used in the constructs in the Examples, as well as other expression signals, are suitable.

### Heterologous Polypeptide

Such a polypeptide can be a binding or target protein, a linker, or an antigen or antibody or part thereof. It is preferably an antigen, and so may be able to elicit or stimulate an immune response. It can be any antigen against which an immune response, more specifically a significant immune response and/or a protective immune response, can be elicited in an animal (preferably a mammal such as a human). The polypeptide (or antigen) may be associated with a pathogen, disease state and/or disorder of the human or animal to which the vaccine is to be administered. Preferably the antigen will be able to interact with one or more (e.g. specific) receptors, for example present on lymphocytes or in antibodies released from them.

The polypeptide can thus comprise an immunogen. Since the polypeptide can be one that can elicit an immune response, this may exclude for example a protein that is present already or originates from in the individual. Antigens are thus preferably foreign to that individual.

Any antigen, antigenic component or epitope known per se that can be expressed in the host can be used as the polypeptide. Usually this will be a peptide, a protein, or an antigenic part or fragment thereof, such as an epitope. As such, it may either be a native antigenic peptide or protein (or part, fragment or epitope thereof) or an antigenic analog or mutant thereof, for instance obtained synthetically or using recombinant DNA techniques.

Recombinant bacteria and/or bacterial strains, as well as vaccines based thereon, may be provided that can be used to illicit a significant immune response, and preferably a protective immune response, against various antigens. Suitable (heterologous) polypeptides thus include:
antigens or antigenic determinants from a virus, bacterium, fungus, yeast or parasite;
allergens;
viral and/or bacterial antigens including those from (e.g. the gp160 envelope protein of) the HIV virus, such as HIV-1 or HIV-2, a surface glycoprotein (of a Leishmania parasite), Shiga-like toxin, Shigella lipopolysaccharide antigen, Escherichia coli heat labile toxin B subunit or K88 or fimbrial antigen, a CFA antigen (of an enterotoxigenic Escherichia coli strain), anthrax toxin, pertussis toxin or toxin from Bordetella pertussis (e.g. P69), tetanus toxin (or a fragment thereof, e.g. TTFC);
antigens from such pathogens as herpes virus, rubella virus, influenza virus, mumps virus, measles virus, polio(myelitis) virus (type 1, 2 or 3), rotavirus, FMDV, respiratory syncytial virus, Campylobacter species, Chlamydial organisms, species of the genus Cryptosporidium, cytomegalovirus, human immunodeficiency virus, Actinomyces species, Entamoeba histolytica, arenaviruses, arboviruses, Clostridium botulinum, species of the genus Candida, Vibrio cholera, Cryptococcus neoformans, EHEC strains of E. coli O157:H7, O26:H11, O111:H8 and O104:H21, ETEC strains of E. coli, strains of E. coli shown to possess enteroinvasiveness (EIEC), EPEC strains of E. coli, EAggEC strains of E. coli., DAEC strains of E coli, filoviridae, parvovirus, Filarioidea, Staphylococcus aureus, species of the genus Clostridium perfringens, C. tetani, V. cholera, N. meningitides, C. trachomatis, Helicobacter pylori, Caliciviruses, Giacardia lamblia, Neisseria gonorrhoeae, hantaviruses, hepatitis viruses types A, B, C, D, E, Legionellae strains, Mycobacterium leprae, Listeria monocytogenes, species of the genus Clostridium perfringens, Borrelia burgdorferi, Pseudomonas pseudoinallei, Epstein Barr virus, Onchocerca volvulus, Poxviruses, Bordetella pertussis, Yersinia pestis, Coxiella burnetti, rabies virus, Treponema pallidium, Mycobacterium tuberculosis, Salmonella typhi, eukaryotic parasite causing malaria, pneumocystis pneumonia, as well as agents causing toxoplasmosis.

Preferably the polypeptide does not comprise a pilus type epitope (e.g. a pilin or adhesintope, such as from P. aeruginosa). The polypeptide may be a combination of two or more of the above linked (e.g. covalently) or as a fusion protein.

Preferably the allergen is a human allergen, or an allergen provoking an allergic reaction in the type or species of individual to whom the composition is to be administered. It may be a house or insect allergen, such as from dust mite, e.g. Der p 1.

Apart from native antigens and antigenic components (including antigenic parts, fragments or epitopes thereof), antigenic mutants or analogues thereof—obtained synthetically or via recombinant DNA techniques—may be used. Also, in the vaccines of the invention, a combination of two or more such antigens may be present or expressed. These antigens may be expressed by a single (type or strain) of bacterial host, or by several different (types or strains) of bacterial host.

Of the above, antigens against and/or specific for rotavirus, respiratory syncytial virus, Mycobacterium tuberculosis, human immunodeficiency virus, E. coli, Vibrio cholera, streptococci and chlamydia are especially preferred for use as an antigen in the vaccines of the invention.

The polypeptide is preferably such that, upon expression, it still allows—at least to some extent, which may be reduced compared to the native strain—the recombinant bacterial host to settle in and/or colonize (part of) the gastrointestinal tract upon administration, and to persist there. This may be for a time sufficient to provide a significant immune response against the antigen and/or the pathogen associated with it.

Preferably the polypeptide is at least 5, 10, 15, 20, 30 or 40 amino acids in length. The polypeptide may be no more than 50, 70, 100 or 200 amino acids long.

### Fusion Proteins

The heterologous polypeptide need not be a single protein, indeed it may be a combination of two or more polypeptides. However, the polypeptides (and hence also the modified protein) is suitably heterologous to the cell in which the (S-layer) protein is expressed. The heterologous polypeptide may therefore be an antigen or toxin linked to a viral protein, for example a tetanus protein (or antigen) covalently linked or fused to a viral protein. Preferably the tetanus protein is TTFC. The viral protein is preferably from a rotavirus, such as of the group Reoviridae. It may be from a rotavirus of group A, B or C. The viral protein may be a capsid protein, for example an inner capsid protein (such a VP6) or an outer capsid protein (such as a VP4 or VP7). The protein may be a viral capsid protein and/or a viral surface protein. It may be a glycoprotein.

Preferably the viral protein is at least 20, 25, 30, 40 to 50 amino acids in length. Preferably it is not a transmembrane protein, or a protein from HIV (such a GP41).

The modified protein may additionally possess an anchor, or an anchor or binding regional domain, in order to allow it to be attached or tethered to a cell surface. This may be necessary if the bacterial cell does not normally express S-layer proteins, for example if it does not possess an S-layer. The anchor may be attached to the C-terminus. For example, if one is to obtain expression in L. casei or L. plantarum, which do not possess an S-layer, then one can employ a cell wall anchor or binding region or domain (such as described earlier for location at the surface of the host), and if necessary a sorting signal, for example from L. casei, such as PrtP (Martinez et al, supra).

### Lactobacillus or LAB Expression Systems

Usually, the expression system will comprise a genetic construct comprising at least one nucleotide sequence encoding the (modified) polypeptide, or antigen(ic component), preferably operably linked to a promoter capable of directing expression of the sequence in the bacterial host. Suitably the polypeptide to be expressed can be encoded by a nucleic acid sequence that is adapted to the preferred codon usage of the bacterial host. The construct may further contain (all) other suitable element(s), including enhancers, transcription initiation sequences, signal sequences, reporter genes, transcription termination sequences, etc., operable in the selected bacterial host.

The construct is preferably in a form suitable for transformation of the bacterial host and/or in a form that can be stably maintained in the bacterial host, such as a vector or plasmid. More preferably, a food grade construct is used.

A particularly preferred construct according to the invention comprises the multi-copy expression vector described in WO-A-96/32487 or WO-A-01/21200, in which the nucleotide sequence encoding the antigen has been incorporated. Such a construct is particularly suitable for expression of a desired protein or polypeptide in a lactic acid bacterium, in particular in a Lactobacillus, at a high level of expression, and also can be used advantageously to direct the expressed product to the surface of the bacterial cell. The constructs may be characterised in that the nucleic acid sequence encoding the antigen is preceded by a 5′ non-translated nucleic acid sequence comprising at least the minimal sequence required for ribosome recognition and RNA stabilisation. This can be followed by a translation initiation codon which may be (immediately) followed by a fragment of at least 5 codons of the 5′ terminal part of the translated nucleic acid sequence of a gene of a lactic acid bacterium or a structural or functional equivalent of the fragment. The fragment may also be controlled by the promoter. The contents of these two WO publications, including the differing embodiments disclosed therein, and all other documents mentioned in this specification, are incorporated herein by reference.

The vaccines of the invention are preferably oral vaccines, that is to say they are adapted for oral administration. Such oral vaccine compositions will usually be alkaline, since usually alkali is required in order to neutralise acid in the stomach, and allow the bacteria (or at least most of them) to pass through the stomach into the intestine alive. It is preferred that most of the bacteria administered will survive the stomach, and pass into the intestine. Increased immune responses can be achievable when the bacteria are alive, that is to say viable, rather than dead. This is because they can continue to express the modified protein in vivo.

If the heterologous polypeptide is inserted at a location where it is not exposed, or at least partly present in the S-layer, then this may reduce or avoid unwanted degradation or proteolytic processing of the polypeptide (in the GI tract). Not all pharmaceutical formulations will be alkaline, and therefore those that are not alkaline (for example nasal formulations) may not be suitable for oral administration.

The sequence encoding the modified protein and/or heterologous polypeptide (e.g. antigen) can be obtained from any natural source and/or can be prepared synthetically using well known DNA synthesis techniques. The sequence encoding the antigen can then (for instance) be incorporated in a suitable expression vector to provide a genetic construct of the invention, which is then used to transform the intended bacterial host strain.

The recombinant bacterial host thus obtained can then be cultured, upon which the harvested cells can be used to formulate the vaccine, optionally after further purification and/or processing steps, such as freeze-drying to form a powder.

The techniques required to create the genetic constructs containing the antigen-encoding sequence and for transforming, culturing and harvesting the bacterial hosts are well known in the art. For instance they are described in standard handbooks, including Sambrook et al, and F. Ausubel et al, eds., “Current protocols in molecular biology”, Green Publishing and Wiley Interscience, New York (1987).

The vaccine comprising the bacterial host can be formulated in a known manner, such as for the formulation of vaccines and/or for the formulation of preparations of live bacteria for oral administration to an animal or human. Reference can be made to preparations for the administration of probiotics, e.g. for the treatment of gastrointestinal disorders.

The vaccine according to the invention can be in a form suitable for oral administration, which may be a solid, semi-solid or liquid form, including but not limited to solutions and/or suspensions of the bacteria, which are usually preferred.

The vaccine preparation may also be in the form of a powder, such as a freeze dried powder that can be reconstituted before use, e.g. using a suitable liquid. It may be in the form of a solid or liquid preparation that is (to be) mixed with solid, semi-solid or liquid food prior to administration. It may also be in the form of a fermented product.

Besides the bacteria, the vaccine may contain one or more pharmaceutically acceptable carriers or excipients, such as water. The vaccine may also contain one or more adjuvants, including immune adjuvants, suitable for oral administration. These are compatible with the bacterial host and suitably do not interfere (too much) with its desired immunogenic properties. According to one embodiment, the adjuvant may be a lactic acid bacterium, such as the bacterial host strain itself, one of the other L. plantarum strains mentioned above, another Lactobacillus species, or even a Lactococcus, Bifidobacterium or Propionibacterium species suitable for oral administration to humans or animals.

Also, the vaccine may contain one or more further therapeutic substances and/or one or more substances that can facilitate and/or enhance the colonization of (part of) the G.I. tract by the bacteria, and/or the growth of the bacteria in the G.I. tract. The preparation may also be in a form suitable for (direct) administration into the stomach or gut, for instance via a tube or catheter. Preferably upon oral administration the bacterial host settles in, and may thereupon colonise, the gastrointestinal tract, or at least a part thereof, such as the mouth, the gut, the small intestine (e.g. duodenum, jejunum or ileum), the large intestine (or part thereof, such as the caecum) or colon, and preferably either the small intestine or the caecum.

### Immunogenicity

The modified protein, and so, if desired, the heterologous polypeptide (e.g. antigen) expressed by the bacterial host thus can come into contact with the mucosal layer, the lining and/or the wall of the G.I. tract (collectively referred to hereinbelow as “wall of the G.I. tract”), and more specifically with cells within this wall. This can mediate an immune response against the antigen(s) thus presented, such as antigen-presenting cells (for example macrophages, dendritic cells and/or B-lymphocytes). This immunological response by the cells within the wall of the G.I. tract may by itself already constitute a significant immune response as defined herein, and/or it may trigger further immunological reactions/responses in the body of the human or animal to which the vaccine has been administered, which again may be a significant response and/or may be a protective response as defined herein. However, the invention is not limited to any specific mechanism via which the recombinant bacterial host elicits any immune response(s). For instance, the immune response elicited by an antigen as expressed by the bacterial host may provide a stronger or enhanced immune response compared to the response that would be elicited by the antigen as such, e.g. as a (free) soluble protein.

The bacterial host—or any part or fragment thereof and/or any further compound(s) produced by it—may interact with the wall of the G.I. tract and/or with specific cells therein such as the cells which mediate and/or are involved in the immune response. This may enable, facilitate or enhance the immune response to the antigens associated with the bacterial host, such as compared to administration, expression and/or use of the antigen as a free, soluble protein.

Although the (modified) protein is preferably expressed by the bacterial host such that the antigen becomes exposed on the cell surface, it is not excluded that—in order to illicit the immune response—the contents of the cells of the bacterial hosts are in situ (i.e. locally at the wall of the G.I. tract) released and/or liberated from the bacterial cell, e.g. by a mechanism which makes the walls of the bacterial cell wall permeable and/or destroys the cells or the bacterial cell wall. Thus it is possible that (in situ at the wall of the G.I. tract) the immunogenic response may not be (just) caused/elicited by the intact bacterial host, but by a part, fragment, fraction or compound thereof for example the antigen as such and/or cell fragments or cell fractions comprising the antigen. Thus although it is preferred that the vaccines comprises intact, viable and/or live bacteria, vaccines that (e.g. also) contain fragments, fractions, lysates etc., of or derived from the recombinant bacterial host are not excluded.

### Dosages

The amount of bacteria administered is not critical, but suitably it is sufficient for the bacteria to settle into and/or colonize (the desired part of) the G.I. tract, and/or to cause a significant immune response. A suitable amount will be at least 10⁸ cfu, preferably 10⁸-10¹⁰ cfu per dose. This may allow a sufficient amount of bacteria to pass into the intestine, if required. Oral administration of doses less than 10⁸ cfu may not always give the desired immunogenicity (at least not in a reliable manner), whereas amounts of more than 5×10¹⁰ cfu, if cumbersome to administer orally, are less preferred. The above amounts of bacteria (dosages) may correspond, to for instance, 10⁶ to 10⁸ an cfu per kg of body weight of the human or animal. The concentration of bacteria in the vaccine (or other formulation) may be at least 5×10⁹/ml, such as at least 10¹⁰/ml. The formulation may be administered for only up to 2, 3 or 4 days. The bacteria may still be detectable in the individual at least 5 days, 7 days or 9 days after the first or last administration.

### Vaccine Formulations

Preferably the individual to be vaccinated is a human or an animal The human can be an infant, immunocompromised person, elderly person or a normally healthy infant, child or adult.

One advantage of the bacterial hosts used is that they can be capable of surviving in/passing through the gut in amounts sufficient to colonize the intestine(s). Nevertheless, one can administer the bacteria in or as a coated or encapsulated preparation, for instance in the form of a delayed release composition or an enteric coated composition. Suitable encapsulating compounds include but are not limited to chitosan, maltodextrin, lipids and oligo- and polysaccharides. Encapsulation may also improve the shelf-life of the vaccine. The vaccine may be adjuvant-free but preferably contains one or more adjuvants.

Strains of other Lactobacillus species may also prove suitable for use as a bacterial host, for example in vaccines of the invention. These can include strains from L. pentosus, L. reuteri, L. animalis (=L. murinus), L. fermentum, L. acidophilus, L. crispatus, L. gasseri, L. johnsonii, L. salivarius, L. brevis, L. rhamnosis, L. helveticus and/or L. paracasei.

The strains useful in the invention preferably have GRAS status and more preferably are food-grade. Also, they are most preferably used in combination with the expression vectors of WO-96/32487 mentioned above, or another vector that gives a level of expression (e.g. intracellularly and/or exposed on the surface of the bacterial host) comparable to this preferred expression vector.

Furthermore, although the use of strains belonging to the genus Lactobacillus are preferred, it is envisaged that suitable strains could possibly also be selected from bifidobacteria and the propriobacteria, e.g. from the genus Bifidobacterium and/or the genus Propriobacterium. Suitable strains can be selected by the skilled person in the same way as L. plantarum and/or Lactobacillus can be selected.

For instance, a suitable test for determining/confirming whether a selected strain is suitable as a bacterial host according to the invention, is to transform the host with a plasmid carrying the replication region of pAMβ1, pNZ71, pLP323 under the control of promoter sequences such as the regulatable promoters Pamy and Pxyl or the constitutive promoters Pldh and Pslpi's. Examples of such vectors are the TTFC carrying vector pPG421 (for surface anchored/surface exposed expression of the TTFC-antigen under control of Pamy) and/or the TTFC carrying vector pPG623 (for intracellular expression of the TTFC-antigen under control of Pxyl), then to administer the recombinant host thus obtained orally to an animal, preferably a mammal (e.g. a mouse such as a BALB/c and/or C57bl/6 mouse), preferably according to the single dose priming and boosting schedule as mentioned in the Examples, followed by measuring the end-point titres of IgG in individual sera by ELISA using tetanus toxoid. In such an assay, the selected recombinant host preferably provides higher (i.e. at least 1% higher) titres than L. plantarum NICMB 8826 and/or L. plantarum 80 when transformed with the same vector and administered under the same conditions; and more preferably titres which are at least 10% higher, even more preferably at least 20% higher.

Where the law allows, the invention also relates to the administration of the bacterium or vaccine to an individual, such as a human or animal (e.g. a mammal), where that individual (or subject where appropriate) is in need of bacterium or vaccine. The individual may require treatment or prophylaxis or a particular disease, as described above.

### Further Variants and Homologues

The (modified) proteins of the invention can if necessary be produced by synthetic means although usually they will be made recombinantly. They may be modified for example by the addition of histidine residues or a T7 tag to assist their identification or purification or by the addition of a signal sequence to promote their secretion from a cell.

Modifications can be made to the original or wild-type sequence, and yet the protein may still be an S-layer protein. This may include the deletion, modification or addition of single amino acids or groups of amino acids within the protein sequence, preferably so that the peptide maintains the basic biological functionality of the protein.

Conservative substitutions may be made, for example according to the following Table Amino acids in the same block in the second column and preferably in the same line in the third column may be substituted for each other. Preferably substitutions do not affect the folding or activity of the polypeptide.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

### Alterations

Proteins of the invention may be chemically altered, e.g. post-translationally. For example, they may be glycosylated (one or more times, by the same or different sugars) or comprise modified amino acid residues. They may also be modified by the addition of histidine residues (to assist their purification) or by the addition of a signal sequence (to promote insertion into the cell membrane). The protein may have one or more (N) amino- or (C) carboxyl-terminal extensions, such as an amino-terminal methionine residue, a small linker peptide of up to about 20-25 residues, or a (small) extension that facilitates purification, such as a poly-histidine or T7 tag, an antigenic epitope or a (e.g. maltose) binding domain (e.g. at the C-terminus). These extensions may or may not be added via a linker,

### Polynucleotides.

The present invention provides an (e.g. isolated and/or purified) polynucleotide encoding a (modified) protein of the invention. The present invention thus provides polynucleotide encoding the modified protein. Also included is a polynucleotide selected from:
(a) a polynucleotide encoding the modified protein or the complement thereof;
(b) a polynucleotide comprising a nucleotide sequence capable of (e.g. selectively) hybridising to a nucleotide sequence set out in (a), or a fragment thereof;
(c) a polynucleotide comprising a nucleotide sequence capable of (e.g. selectively) hybridising to the complement of the nucleotide sequence set out in (a), or a fragment thereof; and/or
(d) a polynucleotide comprising a polynucleotide sequence that is degenerate as a result of the genetic code to a polynucleotide defined in (a), (b) or (c).

Polynucleotides in (b) and (c) can contain the change(s) corresponding to the modification(s) in the protein.

### Hybridisable Sequences

The term “capable of hybridizing” means that the target polynueleotide of the invention can hybridize to the nucleic acid used as a probe (for example the nucleotide sequence of the modified protein, or a fragment thereof or the complement thereof) at a level significantly above background. The nucleotide sequence may be RNA or DNA and thus includes genomic DNA, synthetic DNA or cDNA. Preferably the nucleotide sequence is a DNA sequence and most preferably, a cDNA sequence. Polynucleotides of the invention can be synthesized according to methods well known in the art.

A polynucleotide of the invention can hybridize to the coding sequence or the complement of the coding sequence of SEQ ID No. 1 at a level significantly above background. Background hybridization may occur, for example, because of other cDNAs present in a cDNA library. The signal level (eg. generated by the interaction between a polynucleotide of the invention and the coding sequence or complement of the coding sequence of SEQ No. 1) is typically at least 10 fold, preferably at least 100 fold, as intense as interactions between other polynucleotides and the coding sequence of SEQ ID No. 1. The intensity of interaction may be measured, for example, by radiolabelling the probe, e.g. with ³²P. Selective hybridization may typically be achieved using conditions of low stringency (0.3M sodium chloride and 0.03M sodium citrate at about 40° C.), medium stringency (for example, 0.3M sodium chloride and 0.03M sodium citrate at about 50° C.) or high stringency (for example, 0.3M sodium chloride and 0.03M sodium citrate at about 60° C.). Hybridization may be carried out under any suitable conditions known in the art¹ and, as a guide, low stringency can be 2×SSC at 55° C., medium stringency can be 0.5 to 1.0×SSC at 60° C. and high stringency can be 0.1 or 0.2×SSC at 60° C. or higher (e.g. at 68° C.), all at 0.5% SDS.

### Alterations

Polynucleotides of the invention may comprise DNA or RNA. They may be single or double stranded. They may also be polynucleotides which include within them one or more synthetic or modified nucleotides. A number of different types of modifications to polynucleotides are known in the art. These include methylphosphonate and phosphorothioate backbones and/or addition of acridine or polylysine chains at the 3′ and/or 5′ ends of the molecule. For the purposes of the present invention, it is to be understood that the polynucleotides described herein may be modified by any method available in the art.

It is to be understood that skilled persons may, using routine techniques, make nucleotide substitutions that do not affect the protein sequence encoded by the polynucleotides of the invention to reflect the codon usage of any particular host organism, for example in which the polypeptides of the invention are to be expressed.

The wild-type protein sequence may be modified by nucleotide substitutions, for example from or up to 1, 2 or 3 to 10, 25, 50 or 100 substitutions. The polynucleotide may alternatively or additionally be modified by one or more insertions and/or deletions and/or by an extension at either or both ends. The modified polynucleotide generally encodes a protein which is antigenic or is an S-layer protein. Degenerate substitutions may be made and/or substitutions may be made which would result in a conservative amino acid substitution when the modified sequence is translated, for example as discussed with reference to proteins.

### Recombinant Aspects

The invention also provides vectors comprising a polynucleotide of the invention, including cloning and expression vectors, and methods of growing, transforming or transfecting such vectors in a suitable host cell, for example under conditions in which expression of a polypeptide of the invention occurs. Provided also are host cells comprising a polynucleotide or vector of the invention wherein the polynucleotide is heterologous to the genome of the host cell. The term “heterologous”, usually with respect to the host cell, means that the polynucleotide does not naturally occur in the genome of the host cell or that the polypeptide is not naturally produced by that cell.

Polynucleotides of the invention can be incorporated into a recombinant replicable vector, for example a cloning or expression vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

### Vectors

The polynucleotide of the invention may inserted into an expression cassette. The vector into which the expression cassette or polynucleotide of the invention is inserted may be any vector which may conveniently be subjected to recombinant DNA procedures, and the choice of the vector will often depend on the host cell into which it is to be introduced. Thus, the vector may be an autonomously replicating vector, i.e. a vector which exists as an extra-chromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

Preferably, a polynucleotide of the invention in a vector is operably linked to a regulatory sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence such as a promoter, enhancer or other expression regulation signal “operably linked” to a coding sequence is positioned in such a way that expression of the coding sequence is achieved under condition compatible with the control sequences.

The vector may be a plasmid, cosmid, virus or phage vector, usually provided with an origin of replication, optionally a promoter for the expression of the polynucleotide and optionally an enhancer and/or a regulator of the promoter. A terminator sequence may be present, as may be a polyadenylation sequence. The vector may contain one or more selectable marker genes, for example an ampicillin resistance gene (in the case of a bacterial plasmid) or a neomycin resistance gene (for a mammalian vector). Vectors may be used in vitro, for example for the production of RNA or used to transfect or transform a host cell. They may comprise two or more polynucleotides of the invention, for example for overexpression.

The DNA sequence encoding the protein is preferably introduced into a suitable host as part of an expression cassette (or construct) in which the DNA sequence is operably linked to expression signals which are capable of directing expression of the DNA sequence in the host cells. For transformation of the suitable host with the expression construct transformation procedures are available which are well known to the skilled person. The expression construct can be used for transformation of the host as part of a vector carrying a selectable marker, or the expression construct may be co-transformed as a separate molecule together with the vector carrying a selectable marker. The vector may comprise one or more selectable marker genes.

A vector or expression construct for a given host cell may comprise the following elements operably linked to each other M a consecutive order from the 5′-end to 3′-end relative to the coding strand of the sequence encoding the polypeptide of the first aspect:
(1) a promoter sequence capable of directing transcription of the DNA sequence encoding the polypeptide in the given host cell;
(2) optionally, a signal sequence capable of directing secretion of the polypeptide from the given host cell into a culture medium;
(3) a DNA sequence encoding a mature and preferably active form of the polypeptide;
(4) preferably, a transcription termination region (terminator) capable of terminating transcription downstream of the DNA sequence encoding the polypeptide; and
(5) optionally, a repressor (e.g. XylR).
   Downstream of the DNA sequence encoding the polypeptide there may be a 3′ untranslated region containing one or more transcription termination sites (e.g. a terminator). The origin of the terminator is less critical. The terminator can e.g. be native to the DNA sequence encoding the polypeptide. The terminator may be endogenous to the host cell (in which the DNA sequence encoding the polypeptide is to be expressed).

Enhanced expression of the polynucleotide encoding the protein of the invention may also be achieved by the selection of heterologous regulatory regions, e.g. promoter, secretion leader and/or terminator regions, which may serve to increase expression and, if desired, secretion levels of the protein of interest from the expression host and/or to provide for the inducible control of the expression of the polypeptide of the invention. Preferably at least one, and advantageously all, of these regulatory regions or elements originate from, or are compatible with, Lactobacillus bacteria. This may render the vectors and/or host cells food grade (or GRAS), except for any antigen encoding sequences.

Aside from the promoter native to the gene encoding the protein of the invention, other promoters may be used to direct expression of the polypeptide of the invention. The promoter may be selected for its efficiency in directing the expression of the polypeptide of the invention in the desired expression host.

Promoters/enhancers and other expression regulation signals may be selected to be compatible with the host cell for which the expression vector is designed. For example prokaryotic promoters may be used, in particular those suitable for use in Lactobacillus or E. coli strains.

Examples of bacterial promoters are the xylose, lactate dehydrogenase, α-amylase and SPo2 promoters as well as promoters from extracellular protease genes.

### Host Cells and Expression

In a further aspect the invention provides a process for preparing a polypeptide according to the invention which comprises cultivating a host cell (e.g. transformed or transfected with an expression vector as described above) under conditions to provide for expression (by the vector) of a coding sequence encoding the protein, and optionally recovering the expressed protein. Polynucleotides of the invention can be incorporated into a recombinant replicable vector, e.g. an expression vector. The vector may be used to replicate the nucleic acid in a compatible host cell. Thus in a further embodiment, the invention provides a method of making a polynucleotide of the invention by introducing a polynucleotide of the invention into a replicable vector, introducing the vector into a compatible host cell, and growing the host cell under conditions which bring about the replication of the vector. The vector may be recovered from the host cell. Suitable host cells include bacteria such as Lactobacillus or lactic acid bacteria, E. coli, yeast (e.g. Kluyveromyces), mammalian cell lines and other eukaryotic cell lines, for example insect cells such as Sf9 cells and (e.g. filamentous) fungal cells.

Preferably the polypeptide is produced as a secreted protein in which case the DNA sequence encoding a mature form of the polypeptide in the expression construct is operably linked to a DNA sequence encoding a signal sequence. Preferably the signal sequence is native (homologous) to the DNA sequence encoding the protein. Alternatively the signal sequence is foreign (heterologous) to the DNA sequence encoding the protein, in which case the signal sequence is preferably endogenous to the host cell in which the DNA sequence is expressed.

The vectors may be transconjugated, transformed or transfected into a suitable host cell as described above to provide for expression. This process may comprise culturing a host cell transformed with an expression vector as described above under conditions to provide for expression by the vector of a coding sequence encoding the protein.

A further aspect of the invention thus provides host cells transvonjugated, transformed or transfected with or comprising a polynucleotide or vector of the invention. Preferably the polynucleotide is carried in a vector for the replication and expression of the polynucleotide. The cells will be chosen to be compatible with the said vector and may for example be prokaryotic (for example bacterial), fungal, yeast or plant cells.

The invention encompasses processes for the production of the protein of the invention by means of recombinant expression of a DNA sequence encoding the protein. For this purpose the DNA sequence of the invention can be used for gene amplification and/or exchange of expression signals, such as promoters, secretion signal sequences, in order to allow economic production of the protein in a suitable homologous or heterologous host cell. A homologous host cell is herein defined as a host cell which is of the same species or which is a variant within the same species as the species from which the DNA sequence is derived.

Preferred features and characteristics of one aspect of the invention are equally applicable to another aspect mutatis mutandis.

The invention will now be illustrated by means of the following Examples, which refer to the figures, which are provided merely for the purposes of illustration, and the invention is not to be construed as being limited to the subsequent Examples.

### EXAMPLES

### Summary

The structure of the crystallisation domain, SAN, of the S_{A}-protein of L. acidophilus ATCC 4356 was analysed by insertion and deletion mutagenesis, and by proteolytic treatment. Mutant S_{A}-protein synthesised in E. coli with 7-13 amino acid insertions near the N-terminus or within regions of sequence variation in SAN (amino acid position 7, 45, 114, 125, 193) could form crystalline sheets, whereas insertions in conserved regions or in regions with predicted secondary structure elements (positions 30, 66, 88 and 156) destroyed this capacity. An insertion in the cell wall binding domain (position 345) did not affect crystallisation. FACscan analysis of L. acidophilus synthesising three crystallising and one non-crystallising S_{A}-protein c-myc (19 amino acids) insertion mutant was performed with c-myc antibodies. Fluorescence was most pronounced for insertions at positions 125 and 156, less for position 45 and severely reduced for position 7 Immunofluorescence microscopy revealed a fluorescent ring in a fraction of the bacterial population, suggesting that these bacteria synthesised mutant S_{A}-protein only. The finding that the chromosomal slpA gene was replaced by the mutant allele in a subset of the population corroborates this conclusion. Proteolytic treatment of S_{A}-protein indicated that only sites near the middle of SAN are susceptible, although potential cleavage sites are present through the entire molecule. Expression in E. coli of DNA sequences encoding the two halves of SAN yielded peptides that could dimerise. The results indicate that SAN consists of a ˜12 kDa N- and a ˜18 kDa C-terminal subdomain linked by a surface exposed loop. The capacity of S_{A}-protein of L. acidophilus to present epitopes, up to ˜19 amino acids in length, at the bacterial surface and the exchange of the wild type by a mutant allele in vivo, makes the system suitable for application as an oral delivery vehicle.

### Introduction

Surface layers or S-layers have been found in up to 400 different species of Eu- and Archaebacteria. They consist of one species of (glyco-)protein, the S-protein, which assembles into characteristic two-dimensional crystalline layers at the cell surface. This assembly is an entropy-driven process during which individual 5-protein monomers form multiple interactions with each other and with the under-lying cell envelope (Beveridge, 1994; Sleytr & Messner, 1983). Why certain bacteria possess an S-layer is not always known but they have been shown to function as molecular sieve, scaffold for extracellular enzymes, protective coat or virulence factor (Egelseer et al., 1995; Noonan & Trust, 1997; Sára & Sleytr, 1987).

S-protein structure-function relationships remain poorly understood due to the lack of suitable methods to determine 5-protein structure at the atomic level. Certainly the best understood aspect of S-protein structure is the association of S-proteins with the cell wall. Several cell wall associating domains have been described for S-proteins from Bacillus, Cotynebacterium, Lactobacillus and Thermobacterium. Recent studies have shown that many S-proteins contain one or more copies of the so-called Surface Layer Homology or SLH domain for cell surface attachment (Egelseer et al., 1998; Lemaire et al., 1998; Mesnage et al., 1999; Olabarria et al., 1996). In contrast, much less is known about the role of structural components or domains in S-protein crystal formation but a few studies have shown that distinct crystallisation domains exist in some S-proteins, possibly corresponding to the morphological domains observed in EM studies (Baumeister et al., 1989; Engelhardt & Peters, 1998; Jarosch et al., 2001; Mesnage et al., 2000; Smit et al., 2001).

Several species of the genus Lactobacillus possess an S-layer. The S-layers of L. acidophilus and related species are composed of a single S-protein (S_{A}-protein) of around 45 kDa (Boot et al., 1996). The S_{A}-protein of L. acidophilus ATCC 4356 and CbsA of L. crispatus JCM 5810 are the best studied with respect to structure and function (Boot et al., 1993; Sillanpää et al., 2000; Smit et al., 2001). The function of the S-layer of these organisms is unknown but lactobacillar S-layers may be important for bacterial adhesion to intestinal epithelial cells and extracellular matrix (ECM) components (Schneitz et al., 1993; Toba et al., 1995).

S_{A}-protein of L. acidophilus shows considerable similarity to the putative product of slpB encoded by a silent S-protein gene of L. acidophilus, to S-proteins from L. helveticus and L. crispatus, and to a haem-agglutinating protein (HAP50) from L. acidophilus, but not to other S-proteins (Boot et al., 1996; Boot et al., 1995). Analysis of the primary amino acid sequences of these proteins showed that homology between these proteins is highest in the C-terminal one third of the proteins (77-99% identity) and lower in the N-terminal two third (30-72% identity). While the N-terminal region shows a preponderance of hydrophobic amino acids, the C-terminal region is mainly composed of hydrophilic residues, a large fraction of which consists of basic amino acids, rendering this region highly positively charged.

It has been demonstrated that the N-terminal and C-terminal parts of S_{A}-protein constitute different structural and functional domains. The N-terminal part of S_{A}-protein (amino acids 1-290) constitutes the crystallisation domain, SAN, and is able to form S-layer crystals with lattice parameters similar to those of crystals formed by intact S_{A}-protein. The C-terminal part of S_{A}-protein (amino acids 291-412), SAC, serves to attach the S-layer to the cell wall (Smit et al., 2001).

S_{A}-protein is among the smallest S-proteins known, making S_{A}-protein an ideal candidate for the study of structure-function relationship of S-proteins. Insertion mutagenesis is a method widely used to determine structure-function relationships of proteins (Bingle et al., 1997; Norton et al., 1998; van Geest & Lolkema, 2000; Wong & Hancock, 2000). In the present invention insertion mutagenesis and proteolytic treatment was employed to gain further insight into the structural organisation of the L. acidophilus S-layer protein and its crystallisation domain SAN in particular. The results suggest a structural model for S_{A}-protein in which SAN is comprised of two subdomains of around 15 kDa each flanking a loop region that is exposed to the environment. DNA sequences encoding the two hypothetical subdomains were expressed in E. coli and the properties of the resulting polypeptides were determined.

This shows that functional S_{A}-protein mutants can be used for the efficient surface exposure of a selected epitope making the system suitable for application as an oral vaccination vehicle.

### Results

Insertion of Peptide Linkers in the S_{A}-Protein of L. acidophilus

The linker insertion mutagenesis strategy yielded ten different slpA insertion mutants slpA11, slpA12, slpA13, slpA14, slpA15, slpA16, slpA18, slpA19, slpA20 and slpA21, possessing linker insertions (NcNoX; 7 or 8 amino acids) after amino acid 45, 125, 156, 177, 30, 66, 88, 114, 193 and 349, respectively, of the mature S_{A}-protein. One additional mutant, slpA9c, contained a c-myc epitope insertion at amino acid 7 instead of the NcNoX linker insertion. The mutant genes and unmodified slpA (slpA10), were transferred to expression vector pQE30ΔXN and the corresponding proteins S_{HA9c}; S_{HA11}, S_{HA12}, S_{HA13}, S_{HA14}, S_{HA15}, S_{HA16}, S_{HA18}, S_{HA19}, S_{HA20}, S_{HA21} and S_{HA10} were synthesised in E. coli and purified by metal-affinity chromatography (Table 1).

TABLE 1

| | | | | | |
|---|---|---|---|---|---|
| | Site/type of | Position | | Size | Crystal |
| Mutant | modification | (AA no)¹ | AA composition² | (AA) | formation |

| | | | | | |
|---|---|---|---|---|---|
| 10 | N-terminal histag³ | 0 | -R G S H₆ G S G T-ATTIN | 13 | +++ |
| 11 | BstEII/linker-peptide | 45 | AIAGN-A M A A A R G N-LTGTI | 8 | +++ |
| 12 | AgeI/linker-peptide | 125 | VKFTG-A M A A A R A G-TNSDN | 8 | +++ |
| 13 | MunI/linker-peptide | 156 | TNVSI-T M A A A R G I-ANVYA | 8 | − |
| 14 | SpeI/linker-peptide⁴ | 177 | YDVTS-A M A A A R A S-GATVT | 8 | − |
| 15 | ClaI/linker-peptide | 349 | NAANI-V A M A A A R G-DGTKR | 8 | +++ |
| 16 | PstI/linker-peptide | 30 | PSVSA-S M A A A R G A-VAANT | 8 | − |
| 18 | AlwNI/linker-peptide | 67 | LKADT- M A A A R N T-ENATI | 7 | − |
| 19 | PstI/linker-peptide | 88 | AELAA-S M A A A R G A-GVAYT | 8 | − |
| 20 | StyI/linker-peptide | 114 | KTVTL-A M A A A R G L-GSANS | 8 | +++ |
| 21 | StyI/linker-peptide | 193 | NADNQ-A M A A A R G Q-VNVAN | 8 | + |
| 9c | c-myc | 7 | TTINA-E Q K L I S E E D L N L E-SSSAI | 13 | +++ |
| 11c | c-myc | 45 | -A M V N E Q K L I S E E D L N A R G N- | 19 | +++ |
| 12c | c-myc | 125 | -A M V N E Q K L I S E E D L N A R A G- | 19 | +++ |
| 13c | c-myc | 156 | -T M V N E Q K L I S E E D L N A R G I- | 19 | − |

1: Amino acid (AA) positions relative to the start of the mature S_{A}-protein
2: Planking sequences are underlined: these arc SEQ ID NOS. 3 to 17
3: This His-tag is present at the N-terminus of all mutants listed
4: Insertion of 23 bp instead of 24 bp resulted in a frameshift mutation and truncation of the protein

All mutant genes were expressed at high levels in E. Coli. SDS-PAGE analysis revealed that the gene products had the expected molecular mass (Mr) of about 46 kDa, slightly larger than wild type S_{A}-protein (results not shown). For mutant SHAH a truncated peptide of about 25 kDa was observed, the result of a frame-shift mutation already observed during sequencing. Mutant S_{HA9c} showed a Mr of around 47 kDa due to a larger insert, the c-myc epitope.

Functional Analysis of S_{A}-Protein Mutants Synthesised in E. coli

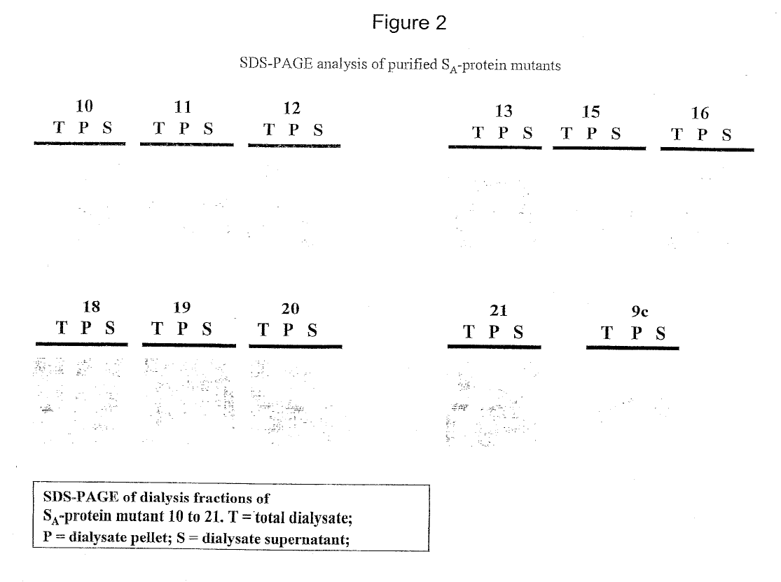

Centrifugation of dialysed mutant S_{A}-proteins allowed discrimination between crystallised and soluble S_{A}-protein. Mutant proteins S_{HA9c}, S_{HA11}, S_{HA12}, S_{HA15}, S_{HA16} and S_{HA20} and the wild type protein S_{HA10} formed a precipitate, indicative of S_{A}-protein crystallisation (Smit et al., 2001), mutant S_{HA21} formed less precipitate than the previous mutants, while mutants S_{HA13}, S_{HA18}, S_{HA19} and the truncated S_{HA14} did not show any precipitate formation. SDS-PAGE analysis of the soluble and insoluble fractions confirmed that a substantial part of S-protein was present in the pellet fractions of S_{HA9c}, S_{HA10}, S_{HA11}, S_{HA12}, S_{HA15}, S_{HA16}, and S_{HA20}. For all other mutants, S_{A}-protein was found only in the supernatant fraction (FIG. 2). Electron microscopical analysis of the precipitates formed by wild type (His-tagged) S_{A}-protein (S_{HA10}) and the precipitating mutants showed crystalline sheets possessing oblique symmetry, similar to that of S_{A}-protein (data not shown).

Three insertion mutants, slpA11, slpA12, and slpA13, were selected for replacement of the NcNoX linker by a c-myc epitope linker. This yielded mutant proteins S_{HA11c}, S_{HA12c} and S_{HA13c} with an insert of 19 amino acids and an Mr of around 47 kDa. Introduction of the larger insert did not alter the properties of the mutant proteins. S_{HA11c} and S_{HA12c} could still form S-layer crystals whereas mutant S_{HA13c} was unable to form crystals as determined by EM (data not shown).

Construction of Expression Vectors for Lactobacillus and Synthesis of S_{A}-Protein with a c-myc Insertion in L. casei and L. acidophilus

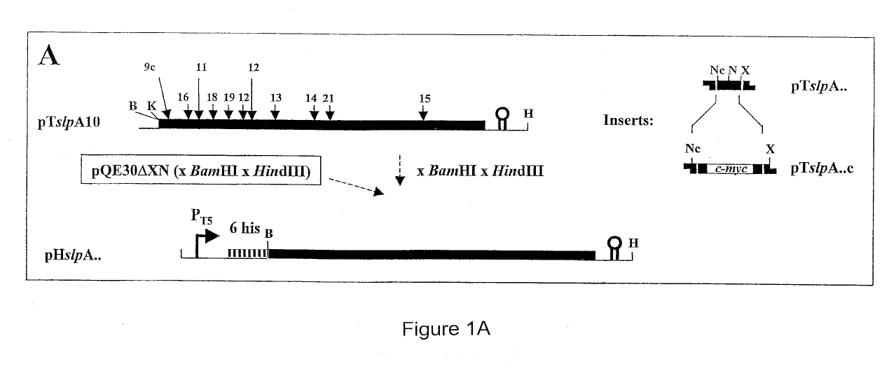

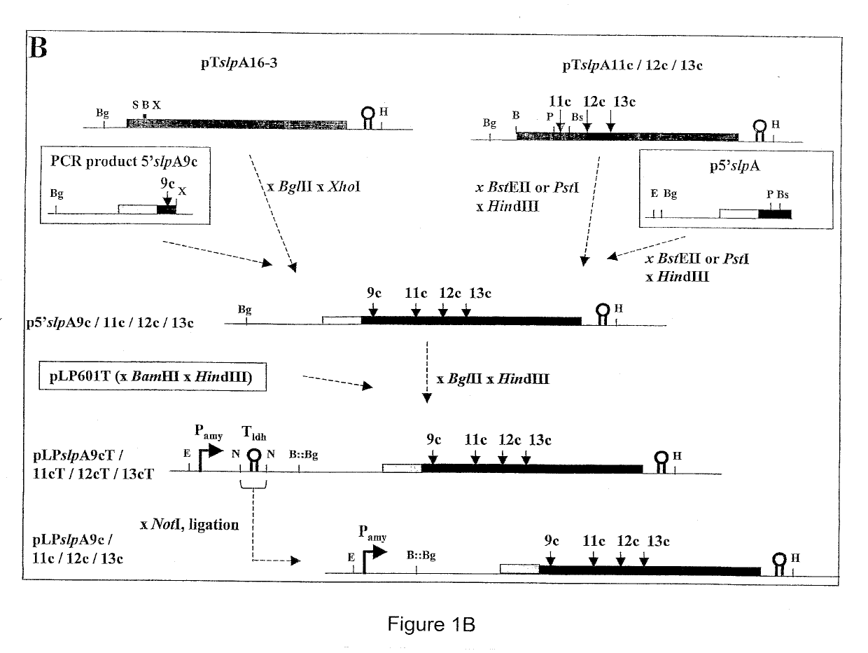

To determine whether the c-myc epitope in mutants S_{HA9c}, S_{HA11c}, S_{HA12}c and S_{HA13c} is buried in the S_{A}-protein interior or is exposed on the S-layer surface, mutant S_{A}-proteins were co-expressed with wild type S_{A}-protein in L. acidophilus. For the expression of mutant S-protein genes in Lactobacillus new vectors were constructed in which the slpA9c, slpA11c, slpA12c and slpA13c genes were combined with the original slpA expression signals (the 5′ leader sequence for mRNA stabilisation, ribosome binding site (RBS), start codon and secretion signal) (FIG. 1). The complete slp cassettes were then transferred to the E. coli-Lactobacillus shuttle vector pLP401-T and introduced into L. casei ATCC 393.

Analysis of mutant slp gene expression in L. casei revealed that all transformants produced mutant S_{A}-protein irrespective of whether the Idh terminator sequence between the promoter and mutant slp gene was present or not (results not shown). Mutant S_{A}-protein production under inducing conditions from vectors without the terminator was only marginally increased compared to that from vectors with the transcription terminator. This constitutive production of mutant S_{A}-protein in L. casei under non-inducing conditions from vectors with the ldh transcription terminator, suggests the presence of a previously undetected promoter sequence in the 5′ region of the slp fragments. In further experiments we used this promoter activity for constitutive mutant S_{A}-protein production in L. casei and L. acidophilus. After confirmation of the synthesis and secretion by L. casei of the mutant S_{A}-proteins, of their correct relative molecular mass and the presence of the c-myc epitope by SDS-PAGE and western analysis (data not shown), CsCl purified vector DNA (without ldh terminator) obtained from L. casei was introduced in L, acidophilus ATCC 4356.

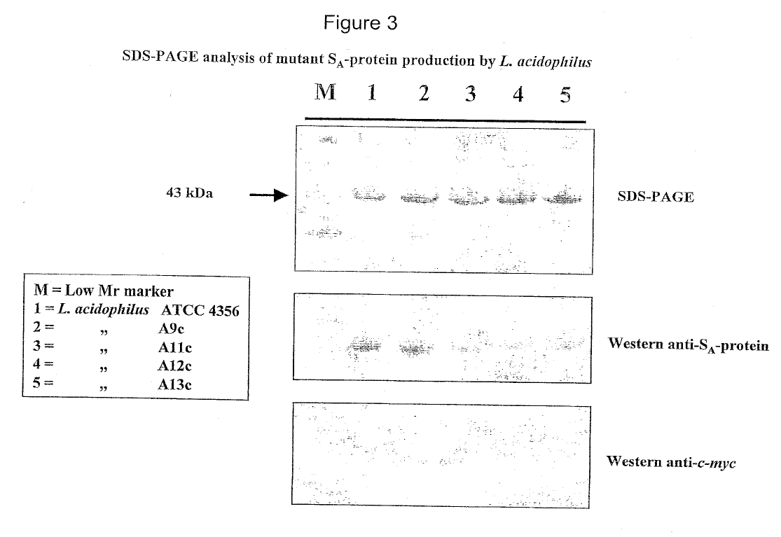

L. acidophilus transformants produced less mutant S_{A}-protein compared to L. casei. From L. acidophilus transformants producing S_{A9c}, S_{A11c}, S_{A12c} and S_{A13c} mutant protein as well as the wild type strain S_{A}-protein could be completely removed by 5 M LiCl extraction, indicating that the bacteria contain a mosaic S-layer comprised of wild type and mutant protein. Mutant proteins were not detected in the culture medium. Using SDS-PAGE and western analysis, an additional band, with a slightly lower mobility than the wild type S_{A}-protein band, was observed in the 5 M LiCl extract of the four L. acidophilus transformants. Western analysis also showed that this band reacted with anti-c-myc antibody while wild type S_{A}-protein did not, confirming the presence of the c-myc epitope (results not shown). It was also observed that mutants S_{A9c} and S_{A11c} were produced at equal levels, while the S_{A12c} production level was lower and that of S_{A13c} even more so (FIG. 3).

SDS-PAGE analysis of soluble and precipitated S_{A}-protein fractions obtained by dialysis and centrifugation of the 5 M LiCl extracts revealed that all four S_{A}-protein mutants associated with both pellet and supernatant fractions, a behaviour typically observed for fully functional wild type S_{A}-protein. No preferential association was observed of the non-crystallising mutant S_{A13c} with the dialysate supernatant fraction although this was observed for the protein purified from E. coli (data not shown).

Exposition of the c-myc Epitope at the Cell Surface of L. acidophilus

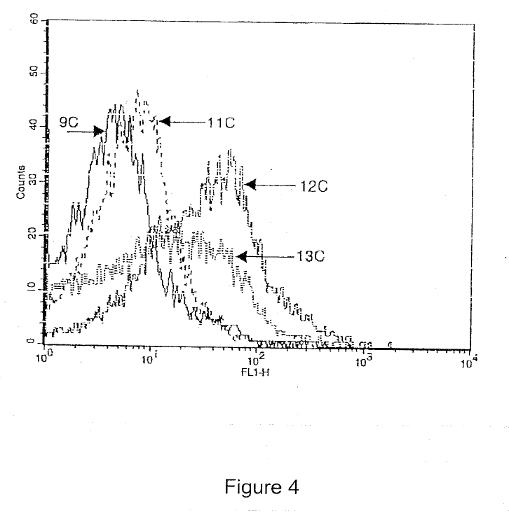

Surface presentation of the c-myc epitope was analysed by flow cytometry and immunofluorescence microscopy. FACScan analysis with c-myc antibodies yielded a signal for each of the L. acidophilus transformants although important differences were observed. The detected fluorescent signal increased in the following order: S_{A9c}, S_{A11c}, S_{A13c} S_{A12c} (FIG. 4). Interestingly, the cytometry signals were not proportional to the mutant protein production levels, which were lowest for the mutants with the highest fluorescent signals (S_{A12c} and S_{A13c}).

The flow cytometry experiments also showed, especially for mutants S_{A12c} and S_{A13c}, a large heterogeneity in fluorescence intensity. Using immunofluorescence microscopy two types of fluorescent cells were observed, one with diffuse fluorescent spots randomly distributed on the cells and another with an intense fluorescent ring completely surrounding the cell (data not shown).

Detection of Recombination at the Chromosomal slpA Locus

The S-layer of cells possessing a surrounding fluorescent ring possibly consists solely of mutant S_{A}-protein since a similar intense ring was observed using wild type cells and anti-S_{A}-protein serum (Boot, 1996). To determine whether the chromosomal copy of the slpA gene in these cells has been inactivated or replaced by the mutant one carried by the expression vector a PCR strategy was applied. Using genomic DNA preparations isolated from L. acidophilus transformants harbouring pLPslpA9c, pLPslpA11c, pLPslpA12c, and pLPslpA13c we were able to amplify recombination-specific fragments for all four transformants, indicating that the chromosomal slpA gene had been replaced by the mutant allele. In a control experiment using wild type L. acidophilus genomic DNA mixed with purified pLPslpA12c as template we did not find any PCR product (data not shown).

### Proteolytic Treatment of S_{A}-Protein

Limited digestion of S_{A}-protein with various proteolytic enzymes yielded one major product of 36 kDa representing SAN, and several smaller fragments. The N-terminal sequence of these fragments has been determined: the fragments obtained after trypsin digestion had the following N-termini ATTIN and VKLDQ (amino acids 1-5 and 140-144), and those obtained after chymotrypsin digestion ATTIN and AINTT (1-5 and 160-164), showing that these peptides represented the N-terminal and C-terminal regions of SAN. Apparently only sites near the middle of SAN are susceptible to proteolytic cleavage although a number of other potential trypsin and chymotrypsin cleavage sites are present in the two peptides.

### Properties of Truncated SAN Fragments

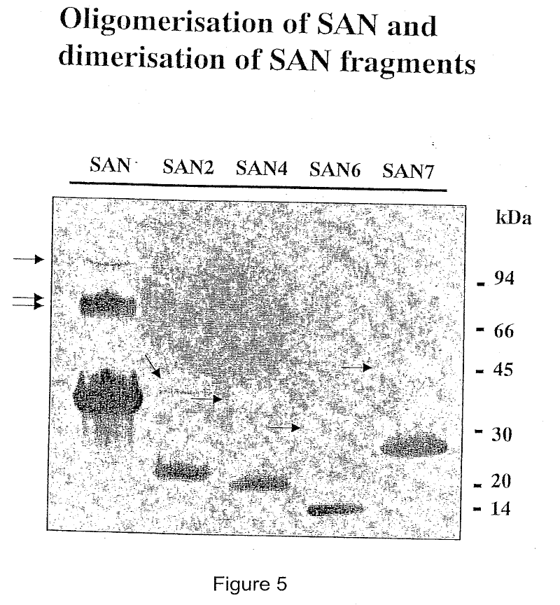

To get further insight in the structure of SAN, vectors were constructed encoding C-terminally (SAN2, 4 and 6 carrying the first 159, 149 and 113 amino acids) or N-terminally truncated peptides (SAN7, 3 and 5 comprising the last 177, 151 and 141 amino acids). The truncated His-tagged genes were expressed in E. coli and the peptides SAN2, SAN4, SAN6 and SAN7 were purified by affinity chromatography. The peptides SAN3 and SAN5 were produced at very low levels and could not be purified probably due to increased sensitivity to E. coli proteases. SDS-PAGE and western analysis indicated that the truncated proteins had the expected size. Western blotting also showed the presence of molecules with twice the size of the monomers for the peptides, SAN2, SAN4, SAN6 and SAN7 (FIG. 5). The stability of the peptides against proteolytic attack and their capacity to dimerise suggests that they constitute functionally active units.

### Discussion

Traditionally, S-layers have been studied by electron microscopical techniques and as a result much is known about their ultrastructure. Little is known, however, about S-protein structure-function relationships. In recent years, with the advent of versatile recombinant DNA technologies, valuable new clues to the structural organisation of S-proteins have been obtained. Methods like deletion analysis, sub-cloning of domains, linker mutagenesis and “cysteine-scanning” mutagenesis have been used for this purpose (Jarosch et al., 2001; Mesnage et al., 1999; Smit et al., 2001).

In the invention linker insertion and deletion mutagenesis were applied as well as S-protein proteolysis to clarify the structural organisation of the crystallisation domain, SAN, of the L. acidophilus S_{A}-protein. The analysis comprised insertion of seven to thirteen amino-acid residues at eleven positions randomly distributed throughout the S_{A}-protein, ten of which were located in SAN. All mutant proteins contained an additional N-terminal His-tag (13 amino acids), which did not interfere with S_{A}-protein crystallisation as was previously confirmed by electron microscopy.

Introduction of insertions at positions 30, 66, 88, and 156 of S_{A}-protein (S_{HA13}, S_{HA16}, S_{HA18} and S_{HA19}) resulted in complete abolishment of the capacity to form crystals in vitro. Mutants with insertions after amino acids 7, 45, 125, 114 (S_{HA9c}, S_{HA11}, S_{HA12}, and S_{HA20}) formed crystals similar to those formed by the His-tagged wild type protein, while an insertion at position 193 (S_{HA21}) resulted in a partially functional mutant S_{A}-protein. An insertion in the cell wall binding domain (S_{HA15}) had no effect on assembly, as expected.

For the interpretation of these results multiple S-protein sequence alignments and predicted S-protein sequence motifs (Sillanpää et al., 2000; Smit et al., 2001) were used. The data obtained from these analyses showed that the SAN domain of S_{A}-protein consists of regions with high variability in composition and length (gap regions) alternating with regions that show considerably less variation. It was proposed that the regions of higher conservation are important for domain structure-function (responsible either for intra- or intermolecular interactions) (Sillanpää et al., 2000; Smit et al., 2001). Disruption of these regions may result in loss of domain function, while regions of variable length are more likely to represent protein surface regions or loops (Miyazawa & Jernigan, 2000) and may accept the insertion of additional amino acid residues.

In general, insertions in regions that were well aligned in S_{A}-protein alignments or contained predicted secondary structure elements were not allowed. This in contrast to insertions in variable, non-conserved regions without any predicted secondary structure elements, which were accepted without disrupting S-layer formation. Two mutated regions are of importance. First the N-terminal region of the S_{A}-protein. The N-terminus itself is flexible and does not directly play a role in S_{A}-protein crystallisation since it accepts an extension of 13 amino acids without loss of domain function (Smit et al., 2001). Insertion of additional amino acids at position 7 (S_{HA9c}) was probably accepted because of the proximity to the S_{A}-protein N-terminus. If the insertion was made further away from the N-terminus (amino acid 30; S_{HA16}) then protein function was compromised, possibly due to improper folding of the region or to interruption of protein-protein interactions between S_{A}-protein monomers. Despite the lack of significant conservation between amino acids 30 and 47, this region seems to be important for S_{A}-protein crystallisation.

The second region in which mutations 12 and 20 were made (positions 125 and 114, respectively), is interesting because of its location in the centre of the SAN domain where insertions of up to 19 amino acids did not affect S_{A}-protein crystallisation. This central region is the largest variable region observed in alignments and is the only region in SAN containing sites that are accessible to proteolytic enzymes.

Finally, flow cytometric and immunofluorescence analysis of mutant S_{A}-protein-producing L. acidophilus showed that the regions near amino acids 125 and 156 (S_{A12c} and S_{A13c}) were strongly reacting with anti-c-myc antibodies, whereas those near amino acids 9 and 45 (S_{A9c} or S_{A11c}) were not. From these results we conclude that the former regions are facing the environment, while the N-terminal region is poorly accessible to antibodies and thus is either buried within SAN or is facing the S-layer pore or cell wall.

Interestingly, mutant S_{A13c}, although non-functional in in vitro analyses, forms an integral part of the S-layer in vivo, since the mutant protein could be extracted from L. acidophilus transformants by 5M LiCl. Moreover, a strong positive signal was observed in the FACS analysis implying that c-myc in S_{A13c} is located at the bacterial surface. Since the S-layer of the transformants is comprised of wild type and mutant S_{A}-protein, we conclude that S_{A13c} can functionally interact with wild type S_{A}-protein.

The difference in distribution of the flow cytometry signals, most clearly detectable for L. acidophilus transformants producing S_{A12c} or S_{A13c}, suggested a difference in mutant protein production levels within the population of bacteria. Immunofluorescence microscopy and PCR experiments showed that in a fraction of the bacterial population the wild type S_{A}-protein was fully replaced by the mutant protein as a result of the exchange of the chromosomal slpA gene by the plasmid encoded mutant allele. Since this phenomenon was also observed for S_{A13c} we assume that S_{A13c} can form a genuine S-layer in vivo. An explanation why S_{A13c} forms an S-layer in vivo but not in vitro might be that interaction of S_{A}-protein with the cell wall stabilises the interaction of the S_{A13c} monomers, or induces a conformational change of the monomers facilitating their assembly. Also the absence of the His-tag in S_{A13c} may positively affect its crystallisation.

Based on the proteolysis experiments it was assumed that the central region divides the SAN domain into two parts that may constitute subdomains. To verify this assumption the properties of N- and C-terminally truncated SAN peptides were determined. Truncated forms of SAN could be produced in E. coli with a size of 12-19 kDa that were insensitive to proteases present in E. coli, when up to 170 C-terminal residues or up to 120 N-terminal residues were deleted. Both C-terminally and N-terminally truncated peptides were capable of forming dimers in vitro that could resist boiling in the presence of 1% SDS. Apparently these fragments represent functionally active units, as they can dimerise through protein-protein interaction under strongly denaturing conditions. Since all three C-terminally truncated peptides were proteolytically stable and could dimerise whereas only the largest N-terminally truncated form (SAN7) showed this behaviour, one can conclude that the subdomain boundaries are located between amino acids 114 and 140.

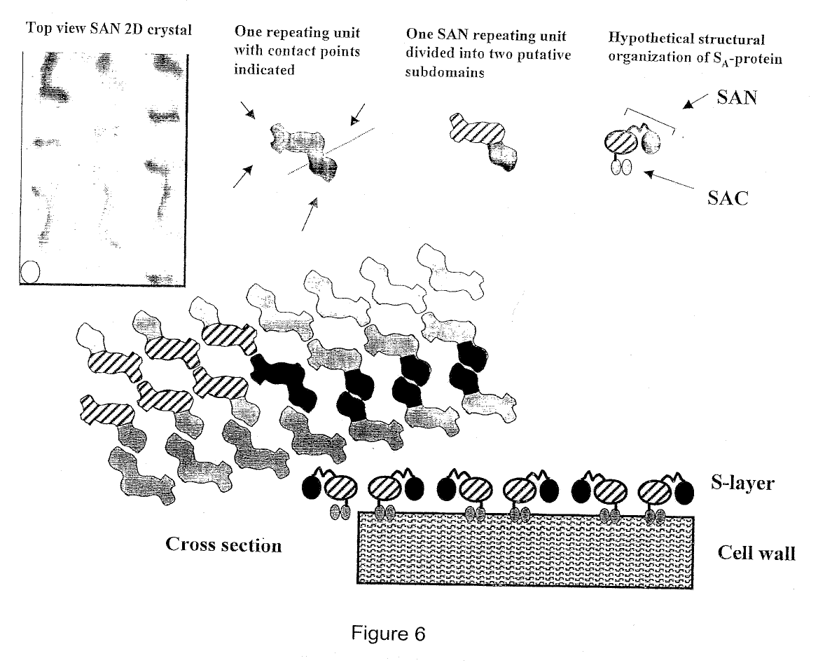

The two subdomains described above could represent the two morphological subdomains observed in our electron crystallographic study of S_{A}-protein and SAN. For each SAN monomer four contact points with neighbouring monomers could be observed, three in the elongated and one in the spherical subdomain (Smit et al., 2001). FIG. 6 shows a model which schematically depicts how crystals of SAN could be formed by dimerisation and oligomerisation of its subdomains.

Finally, the experiments demonstrate how to surface-expose epitopes on the S-layer of L. acidophilus. Based on this finding the presentation of other antigens on the surface of lactobacilli using the S-layer can be used in oral or nasal vaccines. Lactobacilli possess several additional properties that make them highly suitable for such applications, i.e. their GRAS (generally regarded as safe) status, their immunomodulating properties and their capacity to evoke mucosal and systemic immune responses against associated antigens (Maassen et al., 1999; Shaw et al., 2000). The observation that mutant S-layer genes can replace the chromosomal wild type gene by genetic recombination, rendering the mutation genetically stable, further adds to the application potential of genetically engineered Lactobacillus S-proteins.

### Materials and Methods

### Bacterial Strains and Growth Conditions

L. acidophilus ATCC 4356 and L. casei ATCC 393 were cultivated anaerobically in MRS broth (Difco) at 37° C. Lactococcus lactis MG1614 was cultivated aerobically in GM17 medium at 30° C. Escherichia coli M15 (pREP4) (Qiagen) and E. coli DH5α (Phabagen, The Netherlands) were cultivated aerobically in Luria (L) broth at 37° C. When necessary, media were supplemented with 1.5% agarose, 5 μg/ml (L. lactis) or 7.5 μg/ml (L. casei and L. acidophilus) chloramphenicol, 100 μg/ml ampicillin and/or 25 μg/ml kanamycin (E. coli).

Linker Insertion Mutagenesis of the L. acidophilus slpA Gene

A pET5a (Promega) derived vector, pTslpA10 (previously named pTA10), containing a BamHI-HindIII PCR fragment encoding mature S_{A}-protein (Smit et al., 2001) was used for insertion mutagenesis. Double stranded DNA linkers were inserted in unique restriction sites or in sites occurring twice in the slpA gene (FIG. 1a). Linkers consisted of NcoI, NotI and XhoI (NcNoX) sites flanked by restriction site-specific 5′ and 3′ sticky ends. Ligation mixtures were introduced in E. coli DH5α and clones harbouring pTslpA10 vector with the desired linker insert, were identified by colony PCR and analysed by DNA sequencing. The resulting plasmids are indicated with pTslpA followed by the mutant number as indicated in Table 1 (pTslpA11, pTslpA12 etc).

Functional Analysis of S_{A}-Protein Insertion Mutants in E. coli

Mutant S_{A}-protein genes were cloned in pQE30ΔXN as BamHI-HindIII fragments introducing an N-terminal six histidine tag (plasmids are indicated with pHslpA followed by mutant number). Mutant S_{A}-protein gene expression and metal affinity purification were carried out as previously described (Smit et al., 2001). Purified mutant S_{A}-protein (500 μg/ml) was dialysed exhaustively against 50 mM Tris-HCl, pH 7.5 and checked for precipitation, which is indicative of S_{A}-protein crystal formation (Smit et al., 2001). Soluble and precipitated S-protein fractions were separated by centrifugation and analysed by SDS-PAGE. Precipitated material was also analysed by electron microscopy (EM) (Smit et al., 2001). In three mutant plasmids pHslpA11, pHslpA12 and pHslpA13 the NcNoX linker was removed by NcoI and XhoI digestion and replaced by a c-myc epitope linker (NcoI-c-myc-XhoI). The mutant proteins were produced and analysed as described for the other mutants.

Construction of Plasmid pHslpA9c

An additional insertion mutant was constructed by PCR using vector pBK1 (Boot et al., 1993) as template and primers 5′SLPA1 (5′ GCGCGAATTCAGATCTATCGTGGTAAGTAATAGGACGTG 3′: SEQ ID NO. 18) and CMYCRE (5′CAGCGAATTCCTCGAGGTTTAAATCTTCTTCTGAAATTAACTTTTGTTC TGCGTTAATAGTAGTAGCAGCGC 3′: SEQ ID NO. 19) yielding 5′ slpA9c. This PCR product was introduced in vector pTslpA16-3 (containing SalI, BamHI and XhoI sites, after amino acid 7) as BglII-XhoI fragment yielding pT5′ slpA9c. For purification and functional analysis of the mutant S_{A}-protein in E. coli the slp region of pT5′ slpA9c was amplified using primers CEAMYC1 (5′GGGGGGATCCGGTACCGCTACTACTATTAACGCAGAAC 3′: SEQ ID NO. 20) and CEA2 (5′ CCCCGGATCCAAGCTTATCGAAGTATCAGAAGATCCTATT 3′: SEQ ID NO. 21) and the BamHI-BstEII fragment was transferred to pHslpA10 yielding pHslpA9c. Functional analysis of the purified mutant protein was performed as described above.

Re-Introduction of 5′ slpA Expression Signals in Mutants slpA11c, slpA12c and slpA13c

A fragment including the 5′ expression signals of the slpA gene (nt −190 to +150 relative to the ATG), flanked by BglII (5′) and HindIII (3′) sites, was amplified from plasmid pBK1 (Boot et al., 1993) by PCR using primers 5′SLPA1 (5′ GCGCGAATTCAGATCTATCGTGGTAAGTAATAGGACGTG 3′: SEQ ID NO. 22) and 5′SLPA2 (5′ GGGGAAGCTTCAGTAGTGCTACCAGCAGCAG 3′: SEQ ID NO. 23). The PCR product was inserted in pGEM-T and after sequence confirmation excised with EcoRI and HindIII and inserted in pUC19 to give p5′ slpA. Mutant S_{A}-protein cassettes isolated from pTslpA11c, pTslpA12c, and pTslpA13c as BstEII or PstI-HindIII fragments were cloned in p5′ slpA to yield p5′ slpA11c, p5′ slpA12c and p5′ slpA13c. Clones containing the complete slp cassettes were identified by colony PCR and restriction analysis.

Construction of Expression Vectors for Mutant S_{A}-Protein Genes in L. casei and L. acidophilus

To achieve the synthesis of mutant S_{A}-proteins S_{A9c}, S_{A11c}, S_{A12c} and S_{A13c} in L. casei and L. acidophilus, the multi-host range vector pLP401-T was used. This vector contains the inducible promoter of the Lactobacillus amylovorus α-amylase gene (Pouwels et al., 2001). The complete slp cassettes were isolated from pT5′ slpA9c, p5′ slpA11c, p5′ slpA12c and p5′ slpA13c as BglII-HindIII fragments and cloned in BamHI/HindIII digested pLP401-T to yield vectors pLPslpA9c, pLPslpA11c, pLPslpA12c and pLPslpA13c. Ligation mixtures were introduced in L. lactis MG1614 and correct clones were selected by colony PCR. Highly purified plasmid DNA isolates were prepared by CsCl gradient centrifugation, digested with NotI to remove the ldh terminator, ligated and introduced in L. casei ATCC 393. Colony PCR was used to confirm the absence of the terminator sequence.

Expression Analysis of Mutant S_{A}-Protein Genes in L. casei and L. acidophilus

L. casei and L. acidophilus transformants harbouring pLPslpA9c, pLPslpA11c, pLPslpA12c and pLPslpA13c and untransformed L. casei ATCC 393 and L. acidophilus ATCC 4356 were inoculated 1:100 from ON cultures into 50 ml MRS medium and incubated for 16 h at 37° C. Cells were collected by centrifugation (25 min, 3,000×g, 4° C.) and proteins in the culture supernatant were precipitated with TCA. The cell pellet was washed once with physiological salt and subjected to 1 M and 5 M LiCl extraction to remove surface associated proteins as described previously (Smit et al., 2001). The 1 M and 5 M LiCl extracts were dialysed against AD at 4° C.,

Exposition of c-myc at the S-Layer Surface of L. acidophilus Transformants

Exposition of the c-myc epitope on the S-layer surface by L. acidophilus was determined by flow cytometry and immunofluorescence microscopy. Transformants containing pLPslpA9c, pLPslpA11c, pLPslpA12c and pLPslpA13c and wildtype L. acidophilus cells were harvested at the end of the log phase and washed once with phosphate buffered saline (PBS) supplemented with 1% (w/v) fetal calf serum (FCS). After centrifugation the cell pellet was resuspended in PBS-FCS supplemented with 1% FCS containing anti-S_{A}-protein or anti-c-myc (1:5000 and 1:50 diluted, respectively) antibodies. After incubation with FITC-labelled goat-anti-mouse antibodies, cells were analysed with a FACSCalibur flow cytometer (Becton Dickinson, San Jose, Calif.) as described (Pouwels et al., 2001), or inspected with an immunofluorescence microscope.

### PCR Detection of Chromosomal slp Recombinants

Genomic DNA was isolated from transformants containing expression vectors pLPslpA9c, pLPslpA11c, pLPslpA12c and pLPslpA13c. PCR was performed according to standard procedures using primer A9 (forward, slp promoter-specific: 5′-CTTGCTATTTCTTGAAGAG-3′: SEQ ID NO. 24) and primer CMYCINT (reverse, c-myc-insert-specific: 5′-GCGTTTAAATCTTCTTCTGAA-3′: SEQ ID NO: 25) and 15 ng of chromosomal DNA as template. To avoid misinterpretation of the results due to a Polymerase Halt-mediated Linkage Of Primers or PHLOP event, PCR was also performed using purified chromosomal DNA obtained from wild type L. acidophilus which was mixed with purified pLPslpA12c DNA at physiological relevant molar ratios (chrDNA plDNA ratio's of 1:0.3, 1:3, 1:30 and 1:300).

### Construction, Purification and Functional Analysis of Truncated SAN Fragments

Three DNA fragments encoding C-terminally truncated SAN peptides were amplified by PCR with pBK1 (Boot et al., 1993) as template. Primer CEA1 (5′-GGGGGGATCCGGTACCGCTACTACTATTAACGCAAGTTC-3′, forward: SEQ ID NO. 26) was used in combination with primer SANA1 (5′-CCCCGAATTCAAGCTTTTATTAGT-ATACGTTTGCAATTGAAACATTAG-3′, reverse: SEQ ID NO. 27) to amplify SAN2, SANA2 (5′-CCCCAAGCTTTTATTATGAAGCAACACCGTTTTGGTC-3′, reverse: SEQ ID NO. 28) for SAN4 and SANA3 (5′-CCCCAAGCTTTTATTAACCAAGGGTAACAGTCTTACC-3′, reverse: SEQ ID NO. 29) for SAN6. Fragments encoding three N-terminally truncated peptides were amplified using primer SANB1 (5′-GGGGGGATCCGGTACCAAAGTTAAGT-TAGACCAAAACGGTG-3′: SEQ ID NO. 30) for SAN3, SANB2 (5′-GGGGGGATCCGGTACCC-TT-ACTAATGTTTCAATTGCAAACG-3′: SEQ ID NO. 31) for SAN5 and SANB3 (5′-GGGGGG-ATCCGGTACCTCAGCTAACTCAAATGTAAAATT-3′: SEQ ID NO. 32) for SAN7, all three in combination with primer SAN1 (5′CCCCGAATTCAAGCTTTTATTAAATTC-TCTTGCTTAGCTGGGCTAC-3′, reverse: SEQ ID NO. 33) and the same template. The fragments were cloned in pGEM-T and sequenced. The fragments encoding SAN2, SAN3, SAN4, SAN5, SAN6 and SAN7 were excised as BamHI-HindIII fragments and introduced in vector pQE30ΔXN (Smit et al., 2001) in frame with an N-terminal six histidine tag. Expression of gene fragments and purification of the gene products was performed as described previously (Smit et al., 2001). Functional analysis of the truncated SAN peptides consisted of dialysis of samples (500 μg/ml) against 50 mM Tris-HCl, pH 7.5 followed by SDS-PAGE and western analysis.

### DNA Manipulation, Protein Analysis

DNA manipulations and protein analysis were performed following standard procedures (Laemmli, 1970; Sambrook et al., 1989). Plasmid DNA was isolated from Lactococcus/Lactobacillus according to published procedures (Kok et al., 1984; Posno et al., 1991). Genomic DNA was isolated from lactobacilli using cetyltrimethylammonium bromide (CTAB) (Towner, 1991). Transformation of L. lactis, L. casei and L. acidophilus was performed following published procedures (Kok et al., 1984; Posno et al., 1991; Walker et al., 1996). Proteolytic treatment of S_{A}-protein was performed as previously described (Smit et al., 2001).

### Vaccine Preparation and Immunisation

The capacity of recombinant L. acidophilus S-layer proteins to evoke an immune response against the inserted c-myc epitope was assessed for mutant S-protein S_{A12c} that exposes c-myc to the environment and for mutant S-protein S_{A9c} in which c-myc is buried within the S-layer. Female C57B1/6 and Balb/c mice were immunized three times a week with a 3-week interval between immunizations. Purified mutant S-protein (range 25-100 g per dose) in phosphate buffered saline (pH7) or in Freund's incomplete adjuvant (Difco Laboratories) was administered intraperitoneally. Serum samples were collected 14 and 28 days past prime immunisation, and 7 and 14 days past booster immunisation. The induction of antibodies was determined in an ELISA using purified S_{HA9c} S_{HA12c}, and S_{HA10} protein as probes, as well as a purified cmyc carrying recombinant protein (CD81-cmyc) as a positive control. The results indicate the recombinant L. acidophilus S-layer proteins with inserted antigens can evoke an immune response to both the protein itself (Table 2, positive response of all S_{HA} proteins against SA10) as well as to the inserted antigen (Table 2, positive response against CD81-cmyc).

TABLE 2

| | | | | | |
|---|---|---|---|---|---|
| Coat imm | 0 | SA9c | SA10 | SA12c | CD81-cmyc |

Balb/c

| | | | | | |
|---|---|---|---|---|---|
| PBS | − | − | − | − | − |
| SA9c | − | ++ | ++ | ++ | + |
| SA10 | − | ++ | ++ | ++ | − |
| SA12c | − | ++ | ++ | ++ | + |

C57 B1/6

| | | | | | |
|---|---|---|---|---|---|
| PBS | − | − | − | − | − |
| SA9c | − | ++ | ++ | ++ | + |
| SA10 | − | ++ | ++ | ++ | − |
| SA12c | − | ++ | ++ | ++ | − |

The proteins (50 μl) were coated o/n onto plastic wells at 4° C. at a concentration of 0-10 μg/ml and after pretreatment with skimmed milk incubated with anti-cmyc antiserum (Pharmingen). The recombinant protein CD81-cmyc served as a positive control

In a second experiment the capacity of L. acidophilus expressing S_{A12e} or S_{HA9c} to induce antibodies against the c-myc epitope can be determined. 100 g of dead recombinant L. acidophilus in phosphate buffered saline (pH 7) or in Freund's incomplete adjuvant (Difco Laboratories) is to be administered intraperitoneally in a 2501 volume twice with a 4-week interval between immunisations. Serum samples are then collected 14 and 28 days past prime immunisation, and 7 and 14 days past booster immunisation. The induction of antibodies is determined in an ELISA using purified S_{HA9c} protein as probe, as well as with purified c-myc. This can show that recombinant Lactobacilli carrying S-proteins with inserted antigens can be used as vaccine carriers.

### REFERENCES

Baumeister, W., Wildhaber, I. & Phipps, B. M. (1989). Principles of organization in eubacterial and archaebacterial surface proteins. Can. J. Microbiol, 35, 215-227.
Beveridge, T. J. (1994). Bacterial S-layers. Curr. Opin Struct. Biol. 4, 204-212.
Bingle, W. H., Nomellini, J. F. & Smit, J. (1997) Linker mutagenesis of the Caulobacter crescentus S-layer protein: Toward a definition of an N-terminal anchoring region and a C-terminal secretion signal and the potential for heterologous protein secretion. J. Bacteriol. 179, 601-611.
Boot, H. J. (1996). The surface layer protein genes of Lactobacillus acidophilus. Thesis University of Amsterdam.
Boot, H. J., Kolen, C. P. A. M., Pot, B., Kersters, K. & Pouwels, P. H. (1996). The presence of two S-layer protein encoding genes is conserved among species related to Lactobacillus acidophilus. Microbiology 142, 2375-2384.
Boot, H. J., Kolen, C. P. A. M. & Pouwels, P. H. (1995). Identification, cloning and nucleotide sequence of a silent S-layer protein gene of Lactobacillus acidophilus ATCC 4356 which has extensive similarity with the S-layer protein gene of this species. J. Bacteriol. 177, 7222-7230.
Boot, H. J., Kolen, C. P. A. M., van Noort, J. M. & Pouwels, P. H. (1993). S-layer protein of Lactobacillus acidophilus ATCC 4356: purification, expression in Escherichia coli, and nucleotide sequence of the corresponding gene. J. Bacteriol. 175, 6089-6096.
Egelseer, E., Schocher, I., Sára, M. & Sleytr, U. B. (1995). The S-layer from Bacillus stearothermophilus DSM 2358 functions as an adhesion site for a high-molecular-weight amylase. J. Bacteriol. 177, 1444-1451.
Egelseer, E. M., Leitner, K., Jarosch, M., Hotzy, C., Zayni, S., Sleytr, U. B. & Sara, M. (1998). The S-layer proteins of two Bacillus stearothermophilus wild-type strains are bound via their N-terminal region to a secondary cell wall polymer of identical chemical composition. J. Bacteriol. 180, 1488-1495.
Engelhardt, H. & Peters, J. (1998). Structural research on surface layers: a focus on stability, surface layer homology domains, and surface layer-cell wall interactions. J. Struct. Biol. 124, 276-302.
Gilbert, C., Atlan, D., Blanc, B., Portailer, R., Germond, J. E., Lapierre, L. & Mollet, B. (1996). A new cell surface proteinase: sequencing and analysis of the prtB gene from Lactobacillus delbruekii subsp. bulgaricus. J. Bacteriol. 178, 3059-3065.
Howorka, S., Sara, M., Wang, Y., Kuen, B., Sleytr, U. B., Lubitz, W. & Bayley, H. (2000). Surface-accessible residues in the monomeric and assembled forms of a bacterial surface layer protein. J. Biol. Chem. 275, 37876-37886.
Jarosch, M., Egelseer, E. M., Huber, C., Moll, D., Mattanovich, D., Sleytr, U. B. & Sara, M. (2001). Analysis of the structure-function relationship of the S-layer protein SbsC of Bacillus stearothermophilus ATCC 12980 by producing truncated forms. Microbiol. 147, 1353-1363.
Koeks, C. et al (1992) Cell 68 521-531
Kok, J., Van der Vossen, J. & Venema, G. (1984). Construction of plasmid cloning vectors for Lactic streptococci which also replicate in Bacillus subtilis and Escherichia coli. Appl. Environm. Microbiol. 48, 726-731.
Laemmli, U. K. (1970). Cleavage of structural proteins during the assembly of the head of bacteriophage T4. Nature 227, 680-685.
Lemaire, M., Miras, I., Gounon, P. & Beguin, P. (1998). Identification of a region responsible for binding to the cell wall within the S-layer protein of Clostridium thermocellum. Microbiology 144, 211-217.
Maassen, C. B., Laman, J. D., den Bak-Glashouwer, M. J., Tielen, F. J., van Holten-Neelen, J. C., Hoogteijling, L., Antonissen, C., Leer, R. J., Pouwels, P. H., Boersma, W. J. & Shaw, D. M. (1999). Instruments for oral disease-intervention strategies: recombinant Lactobacillus casei expressing tetanus toxin fragment C for vaccination or myelin proteins for oral tolerance induction in multiple sclerosis. Vaccine 17, 2117-2128.
Mesnage, S., Fontaine, T., Mignot, T., Delepierre, M., Mock, M. & Fouet, A. (2000). Bacterial SLH domain proteins are non-covalently anchored to the cell surface via a conserved mechanism involving wall polysaccharide pyruvylation. EMBO J. 19, 4473-4484.
Mesnage, S., Tosi-Couture, E., Mock, M. & Fouet, A. (1999). The S-layer homology domain as a means for anchoring heterologous proteins on the cell surface of Bacillus anthracis. J. Appl. Microbiol. 87, 256-260.
Miyazawa, S. & Jernigan, R. L. (2000). Identifying sequence-structure pairs undetected by sequence alignments. Prot. Eng. 13, 459-475.
Navarre, W. W. (1994) Proteolytic cleavage and cell wall anchoring at the LPXTGX motif of surface proteins in Gram-positive bacteria, Mol. Microbiol. 14 115-121.
Noonan, B. & Trust, T. J. (1997). The synthesis, secretion and role in virulence of the paracrystalline surface protein layers of Aeromonas salinonicida and A. hydrophila. FEMS Microbiol. Lett, 154, 1-7.
Norton, D. D., Dwyer, D. S. & Muggeridge, M. I. (1998). Use of a neural network secondary structure prediction to define targets for mutagenesis of herpes simplex virus glycoprotein B. Virus Res. 55, 37-48.
Olabarria, G., Carrascosa, J., L., Pedro, M., A., de, & Berenguer, J. (1996). A conserved motif in S-layer proteins is involved in peptidoglycan binding in Thermus thermophilus. J. Bacteriol. 178, 4765-4772.
Pederson, J. A., Mileski, G. J., Weimer, B. C. & Steele, J. L. (1999). Genetic characterization of a cell envelope-associated proteinase from Lactobacillus helveticus CNRZ32. J. Bacteriol. 181, 4592-4597.
Posno, M., Leer, R. J., van Lujk, N., van Giezen, M. J. F., Heuvelmans, P. T. H. M., Lokman, B. C. & Pouwels, P. H. (1991). Incompatibility of Lactobacillus vectors with replicons derived from small cryptic Lactobacillus plasmids and segregational instability of the introduced vectors. Appl. Environm. Microbiol. 57, 1822-1828.
Pouwels, P. H., Vriesema, A., Martinez, B., Tielen, F. J., Seegers, J. F. M. L., Leer, R. J., lore, J. & Smit, E. (2001). Lactobacilli as vehicles for targeting antigens to mucosal tissues by surface exposition of antigens. Meth. Enzymol. 336, 369-389.
Pugsley, A. P. (1993) The complete general secretory pathway in gram-negative bacteria. Microbiological Reviews 57, 50-108.
Sambrook, J., Fritsch, E. F. & Maniatis, T. (1989). Molecular Cloning: A Laboratory Manual. 2nd edit, Cold Spring Harbor Laboratory Press, Cold Spring Harbor, N.Y.
Sára, M. & Sleytr, U. B. (1987). Molecular sieving through S-layers of Bacillus stearothermopilus strains. J. Bacteriol. 169, 4092-4098.
Schneitz, C., Nuotio, L. & Lounatma, K. (1993). Adhesion of Lactobacillus acidophilus to avian intestinal epithelial cells mediated by the crystalline bacterial cell surface layer (S-layer). J. Appl. Bacteriol. 74, 290-294.
Shaw, D. M., Gaerthé, B., Leer, R. J., van der Stap, J. G. M. M., Smittenaar, C., Heijne den Bak-Glashouwer, M.-J., Thole, J. E. R., Tielen, F. J., Pouwels, P. H. & Havenith, C. E. (2000). Engineering the microflora to vaccinate the mucosa: serum immunoglobulin G responses and activated draining cervical lymph nodes following mucosal application of tetanus toxin fragment C-expressing lactobacilli. Immunol. 100, 510-518.
Sillanpää, J., Martinez, B., Antikainen, J., Toba, T., Kalkkinen, N., Tankka, S., Lounatmaa, K., Keränen, J., Höök, M., Westerlund-Wikström, B., Pouwels, P. H. & and Korhonen, T. K. (2000). Characterization of the collagen-binding S-layer protein CbsA of Lactobacillus crispatus. J. Bacteriol 182, 6440-6450.
Sleytr, U. B. & Messner, P. (1983). Crystalline surface layers on bacteria. Annu. Rev. Microbial. 37, 311-339.
Smit, E., Oling, F., Demel, R., Martinez, B. & Pouwels, P. H. (2001). The S-layer Protein of Lactobacillus acidophilus ATCC 4356: Identification and Characterisation of Domains Responsible for S-protein Assembly and Cell Wall Binding. J. Mol. Biol. 305, 245-257.
Toba, T., Virkola, R., Westerlund, B., Björkman, Y., Sillanpää, J., Vartio, T., Kalkkinen, N. & Korhonen, T. K. (1995). A collagen-binding S-layer protein in Lactobacillus crispatus. Appl. Environm. Microbiol. 61, 2467-2471,
Towner, P. (1991) Essential Molecular Biology: A practical Approach, 54-55. (Brown, T. A., Ed.), Oxford University Press, Oxford.
van Geest, M. & Lolkema, J. S. (2000). Membrane topology of the Na(+)/citrate transporter CitS of Klebsiella pneumoniae by insertion mutagenesis. Biochim. Biophys. Acta 1466, 328-338.
Walker, D. C., Aoyama, K. & Klaenhammer, T. R. (1996). Electrotransformation of lactobacillus acidophilus group A1. FEMS Microbiol. Lett. 138, 233-237.
Wong, K. K. & Hancock, R. E. (2000). Insertion mutagenesis and membrane topology model of the Pseudomonas aeruginosa outer membrane protein OprM. J. Bacteriol. 182, 2402-2410.

What is claimed is:

1. A modified bacterial surface layer (S-layer) protein, the modification comprising the internal insertion of a heterologous polypeptide, wherein said unmodified protein is:
(a) able to crystallize to form a crystalline monolayer,
(b) from a Lactobacillus bacterium,
(c) from 40 to 70 kd in size, and
(d) highly basic with a pI of at least 9;
where the insertion site of said heterologous polypeptide is:
(i) at a position from amino acids 1 to 20,
(ii) at a position from amino acids 35 to 55,
(iii) at a position from amino acids 100 to 130,
(iv) at a position from amino acids 110 to 140,
(v) at the position of amino acid 193, and/or
(vi) at a position from amino acids 340 to 360.

2. A protein according to claim 1 wherein the heterologous polypeptide is a functional polypeptide or a polypeptide of interest.

3. A protein according to claim 1 wherein the protein retains most of the full length sequence of the unmodified S-layer protein.

4. A protein according to claim 1 which:
(e) has a crystallization or N-terminal domain that is predominantly hydrophobic, or
(f) has a C-terminal domain which is either predominantly basic or hydrophilic, or
(g) has alternating hydrophobic and hydrophilic regions.

5. A protein according to claim 1 wherein the heterologous polypeptide is inserted at a location in the protein so that it is:
(h) exposed or present on the cell surface, or
(i) present in the surface layer or the cell wall, or
(j) is protected from external proteolytic processing or is not recognized or bound by external antibodies.

6. A protein according to claim 1 wherein the modified or unmodified protein:
(k) crystallizes, optionally into an oblique lattice and/or
(l) has a cell wall anchor domain.

7. A protein according to claim 1 wherein the polypeptide comprises an antigen causing or specific for a disease, and optionally is an antigen recognizable by an antibody or is all or part of an antigen from an anaerobic bacteria, optionally Clostridium.

8. A protein according to claim 1 which in unmodified form is from Lactobacillus acidophilus, L. crispatus, L. helveticus, L. amylovorus, or L. gallinarum.

9. A fragment of a protein according to claim 1 which is:
a) an N-terminal fragment, or a fragment that is capable of forming a dimer with another such fragment or a trimer with two other such fragments and
b) capable of forming dimers with another such fragment and either
(i) includes an immunodominant or exposed loop region and is from 20 to 200 amino acids long or
(ii) excludes an entire immunodominant or exposed loop region and is from 20 to 155 amino acids long.

10. A polynucleotide encoding a protein according to claim 1.

11. A vector comprising a polynucleotide according to claim 10.

12. A host cell comprising, or which is has been transformed with a vector according to claim 11.

13. A bacteria expressing a surface layer (S-layer) protein according to claim 1 or a fragment thereof which is:
a) an N-terminal fragment, or a fragment that is capable of forming a dimer with another such fragment or a trimer with two other such fragments and
b) capable of forming dimers with another such fragment and either;
(i) includes an immunodominant or exposed loop region and is from 20 to 200 amino acids long or
(ii) excludes an entire immunodominant loop region and is from 20 to 155 amino acids long.

14. A bacteria according to claim 13 which is a lactic acid bacteria.

15. A modified bacteria which has been modified to express a heterologous surface layer (S-layer) protein, wherein said heterologous surface layer (S-layer) protein is a modified bacterial surface layer (S-layer) protein according to claim 1.

16. A bacteria according to claim 15 which would not normally, or as a wild-type or in unmodified form does not, possess a surface layer.

17. A modified bacteria according to claim 15 which is a Lactobacillus cell and/or the S-layer has its own, original, cell wall anchor.

18. A bacteria according to claim 15 which is a Lactobacillus bacterial cell, and/or the S-layer protein is from Lactobacillus bacteria.

19. An L. casei bacterial cell expressing a bacterial surface layer (S-layer) protein that is either not from L. crispatus or is not a collagen binding protein.

20. An L. casei cell according to claim 19 wherein the S-layer protein is, or is derived, from L. acidophilus.

21. A modified bacteria expressing only, or homogeneously, a heterologous, or modified surface layer (S-layer) protein according to claim 1.

22. A bacteria according to claim 21 having a genome which includes a polynucleotide encoding a heterologous S-layer protein, and/or where the polynucleotide encoding the normal or wild-type S-layer protein has been silenced, replaced, switched off, or otherwise rendered non-expressed.

23. A bacteria according to claim 22 wherein the modified S-layer protein is the sole or only S-layer protein expressed by the bacterial cell and/or the cell does not express any wild-type S-layer protein.

24. A bacteria according to claim 22 wherein the S-layer protein is located on the surface of the cell wall and/or a multiplicity of S-layer proteins form an S-layer.

25. A vaccine comprising a bacteria according to claim 13, a modified bacteria which has been modified to express a heterologous surface layer (S-layer) protein, wherein said heterologous surface layer (S-layer) protein is a modified bacterial surface layer (S-layer) or a modified bacteria expressing only, or homogeneously, a modified surface layer (S-layer) protein, wherein said bacteria has GRAS (generally regarded as safe) status.

26. A vaccine according to claim 25 which is an oral or nasal vaccine and/or additionally comprises an adjuvant.

27. A sheet or monolayer or 2-dimensional array comprising a plurality of bacterial surface layer proteins, at least one of which is modified protein according to claim 1.

28. A solid surface, liquid-air interface, lipid film, liposome, or solution comprising a sheet, monolayer, or array according to claim 27.

29. A solid surface according to claim 28 to which is bound one or more (macro) molecules, such as an enzyme, antibody or other binding molecule, receptor, antigen, or ligand.

30. A solid surface comprising a layer of S-proteins, at least a plurality of which are modified proteins according to claim 1, which are sandwiched between the surface and a layer of functional molecules.

31. A sensor, molecular sieve or ion trap comprising a sheet, layer or array according to claim 27 or a surface comprising a sheet, monolayer, or array.

32. A sensor, molecular sieve or ion trap comprising a solid surface comprising a layer of S-proteins, at least a plurality of which are modified proteins according to claim 1, which are sandwiched between the surface and a layer of functional molecules.

33. A protein according to claim 1, wherein said modified protein is able to form a sheet, crystalline monolayer, or two-dimensional array.

34. A protein according to claim 8 wherein the modified protein is from Lactobacillus acidophilus.

35. A protein according to claim 34 wherein the heterologous polypeptide is inserted at a location:
(i) from amino acid 1 to amino acid 20 of SEQ ID NO: 2,
(ii) from amino acid 35 to amino acid 55 of SEQ ID NO: 2,
(iii) from amino acid 100 to amino acid 130 of SEQ ID NO: 2, and/or
(iv) from amino acid 110 to amino acid 140 of SEQ ID NO: 2.

36. A protein according to claim 35 wherein the heterologous polypeptide is inserted at a location:
(i) from amino acid 5 to amino acid 10 of SEQ ID NO: 2,
(ii) from amino acid 40 to amino acid 50 of SEQ ID NO: 2,
(iii) from amino acid 110 to amino acid 120 of SEQ ID NO: 2, and/or
(iv) from amino acid 120 to amino acid 130 of SEQ ID NO: 2.

37. A protein according to claim 36 wherein the heterologous polypeptide is inserted at position 7, 45, 114, and/or 125 of SEQ ID NO: 2.

38. A method of using a protein according to claim 1, said method comprising administering the modified protein or a bacteria expressing the modified protein to a human or animal by mucosal, basal, oral, or vaginal delivery.

39. A method of making a protein according to claim 1, said method comprising cultivating a host cell under conditions to provide for expression of the proteins and recovering the expressed protein.

40. A modified bacterial surface layer (S-layer) protein, the modification comprising the internal insertion of a heterologous polypeptide, wherein said unmodified protein is:
(a) able to crystallize to form a crystalline monolayer,
(b) from a Lactobacillus bacterium,
(c) from 40 to 70 kd in size, and
(d) highly basic with a pI of at least 9;
where the insertion site of said heterologous polypeptide is:
(i) at a position from amino acids 1 to 20,
(ii) at a position from amino acids 35 to 55,
(iii) at a position from amino acids 100 to 130,
(iv) at a position from amino acids 110 to 140,
(v) at the position of amino acid 193, and/or
(vi) at a position from amino acids 320 to 410.
